SEQUENCE LISTING (2 sequences)

SEQ ID NO: 1 - protein, 392 aa, Aspergillus niger
MAPKSLFYSLFSTISVALASSIPQTDYDVIVVGGGPAGLSVLSSLGRMRRKTVMFDSGEYRNGVTREMHDVLGFDGTPPAQFRGLARQQISKYNSTSVIDIKIDSITPVEDAAANSSYFRAVDANGTQYTSRKVVLGTGLVDVIPDVPGLREAWGKGIWWCPWCDGYEHRDEPLGILGGLPDVVGSVMETHTLYSDIIAFTNGTYTPANEVALAAKYPNWKQQLEAWNVGIDNRSIASIERLQDGDDHRDDTGRQYDIFRVHFTDGSSVVRNTFITNYPTAQRSTLPEELSLVMVDNKIDTTDYTGMRTSLSGVYAVGDCNSDGSTNVPHAMFSGKRAGVYVHVEMSREESNAAISKRDFDRRALEKQTERMVGNEMEDLWKRVLENHHRRS

SEQ ID NO: 2 - protein, 561 aa, Hypocrea jecorina
MLLSASLSALALATVSLAQGTTHIPVTGVPVSPGAAVPLRQNINDLAKSGPQWDLYVQAMYNMSKMDSHDPYSFFQIAGIHGAPYIEYNKAGAKSGDGWLGYCPHGEDLFISWHRPYVLLFEQALVSVAKGIANSYPPSVRAKYQAAAASLRAPYWDWAADSSVPAVTVPQTLKINVPSGSSTKTVDYTNPLKTYYFPRMSLTGSYGEFTGGGNDHTVRCAASKQSYPATANSNLAARPYKSWIYDVLTNSQNFADFASTSGPGINVEQIHNAIHWDGACGSQFLAPDYSGFDPLFFMHHAQVDRMWAFWEAIMPSSPLFTASYKGQSRFNSKSGSTITPDSPLQPFYQANGKFHTSNTVKSIQGMGYSYQGIEYWQKSQAQIKSSVTTIINQLYGPNSGKKRNAPRDFLSDIVTDVENLIKTRYFAKISVNVTEVTVRPAEINVYVGGQKAGSLIVMKLPAEGTVNGGFTIDNPMQSILHGGLRNAVQAFTEDIEVEILSKDGQAIPLETVPSLSIDLEVANVTLPSALDQLPKYGQRSRHRAKAAQRGHRFAVPHIPPL

(19) United States
(12) Patent Application Publication
(10) Pub. No.: US 2011/0123676 A1
(43) Pub. Date: May 26, 2011

(54) ENZYMATIC TREATMENT OF A PROTEINACEOUS SUBSTRATE BY ENZYMATIC REMOVAL OF FREE THIOLS

(76) Inventors: Peter Edvard Degn, Ega (DK); Jesper Kampp, Skanderborg (DK); Liv Spångner Christiansen, Frederiksberg (DK)

(21) Appl. No.: 12/875,206

(22) Filed: Sep. 3, 2010

Related U.S. Application Data
(63) Continuation-in-part of application No. PCT/EP2009/052558, filed on Mar. 4, 2009.
(60) Provisional application No. 61/033,612, filed on Mar. 4, 2008.

(30) Foreign Application Priority Data
Mar. 4, 2008 (DK) PA 2008 00320

(51) Int. Cl. A23L 1/31; A23J 3/34; A23J 1/02; C12P 21/02; A23L 1/325; A23J 1/04

(52) U.S. Cl. 426/56; 426/7; 426/657; 435/68.1

(57) ABSTRACT

The invention relates to the enzymatic treatment of a proteinaceous substrate with a first enzyme, such as a sulfhydryl oxidase. The first enzyme removes enzyme inhibitors, such as free thiols, present in the proteinaceous substrate. The removal of the inhibitory compounds in the substrate allows for an effective enzymatic action of a second enzyme such as protein cross-linking of the protein present in the substrate by tyrosinase enzymes.

### INCORPORATION BY REFERENCE

This application is a continuation-in-part application of international patent application Serial No. PCT/EP2009/052558 filed 4 Mar. 2009, which published as PCT Publication No. WO 2009/109602 on 11 Sep. 2009, which claims benefit of U.S. provisional patent application Ser. No. 611,033,612 filed 4 Mar. 2008 and Danish patent application Serial No. PA 2008 00320 filed 4 Mar. 2008.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The invention relates to the enzymatic treatment of proteinaceous substrates with a first enzyme, such as a sulfhydryl oxidase. The first enzyme removes enzyme inhibitors, such as free thiols, present in the proteinaceous substrate. The removal of the inhibitory compounds in the substrate allows for an effective enzymatic action of a second enzyme such as protein cross-linking of the protein present in the substrate by a tyrosinase.

### BACKGROUND OF THE INVENTION

WO02/14484 and WO02/14595 relate to a tyrosinase enzyme isolated from Pseudomonadaceae, and its use for protein cross-linking, particularly of protein fibres which as wool fibres.

WO2006/084953 relate to a tyrosinase enzyme obtainable from Trichoderma spp. and its use in cross-linking food proteins.

Sulfhydryl oxidases catalyze the oxidation of sulfhydryl groups to disulfides, according to the general reaction: 2RSH+O2→RS—SR+H₂O₂

Numerous sulfhydryl oxidases are known in the art for example: Sulfhydryl oxidases derived from yeast are available commercially, for example from X-Zyme (Erv1p and Erv1p-X1) GmbH (Duesseldorf/Germany) (http://www.x-zyme.com/de/products.html); R. S. de la Motte and F. W. Wagner, Biochemistry 26 (1987)7363-7371 provides a sulfhydryl oxidase from Aspergillus niger; U.S. Pat. No. 4,087,328 reports on a sulfhydryl oxidase prepared from milk; U.S. Pat. No. 4,632,905 reports on a sulfhydryl oxidase obtained from Aspergillus sojae; U.S. Pat. No. 4,894,340 reports on a sulfhydryl oxidase from Aspergillus niger; Hoober et al., 1996, Journal of Biological Chemistry 271 No 48 pp 30510-30516 provides a chicken egg white sulfhydryl oxidase.

The use of sulfhydryl oxidases in food preparation is known, for example for use in baking: according to EP 0 321 811B, sulfhydryl oxidases may be used to strengthen dough, apparently in combination with a glucose oxidase. According to, EP 0 705 538B bovine and microbial sulfhydryl oxidases may be used as an enzyme additive for dough in the combination with a hemicellulose. WO 2006/046146 indicates sulfhydryl oxidase may be used in the preparation of a durum wheat flour baked product.

WO 2007/093674 relates to a method of preparing a low-ingredient meat product by adding tyrosinase, and to a low-ingredient meat product modified by tyrosinase. The tyrosinase is used to modify texture or water binding properties of low-ingredient meat products which have a low content of at least salt, phosphate or meat.

Lantto et al., LWT 389 (2006) 1117-1124 pertains to the application of a microbial transglutaminase, mushroom tyrosinase and apple powder containing both polyphenol oxidase and transglutaminase, in pork meat. According to Lantto et al., all enzyme preparations were able to improve gel hardness of unheated meat homogenates. Added cysteine affected positively on hardness of apple powder treated pork meat but negatively on the mushroom tyrosinase and microbial transglutaminase treated meats.

Citation or identification of any document in this application is not an admission that such document is available as prior art to the present invention.

### SUMMARY OF THE INVENTION

The present invention is based upon the discovery that inhibitory agents, such as free thiols, which are naturally present in proteinaceous substrates, such as proteinaceous foodstuffs, are responsible for the inhibition of several enzymes such as tyrosinases, and that the use of a first enzyme, which may be capable of reducing or eliminating such agents, such as sulfhydryl oxidases, allow enzymes such as tyrosinases inhibited by inhibitory agents to be utilised effectively for example to cross link protein present in the proteinaceous substrate, which may improve the gel strength, water holding capacity and/or improve the texture of the proteinaceous substrate.

In one aspect, the invention relates to the treatment of proteinaceous substrates with a first enzyme, such as a sulfhydryl oxidase prior to, or simultaneous with treatment with a second enzyme, the activity of which is inhibited by free thiols. The first enzyme removes enzyme inhibitors, such as free thiols, present in the proteinaceous substrate. The removal of the inhibitory compounds in the substrate may allow for a more effective action of the second enzyme, such as protein cross-linking of the protein present in the substrate by tyrosinase enzymes.

The invention provides in another aspect for a method for the preparation of a cross-linked proteinaceous substrate, said method comprising the steps:

a. treatment of a proteinaceous substrate comprising free thiols with a first enzyme capable of removing free thiols;

b. treatment of the proteinaceous substrate with a second enzyme, the activity of which is inhibited by free thiols;

wherein step a) is performed prior to, or simultaneous to step b), and wherein step b) results in the cross-linking of proteins present in said proteinaceous substrate.

The invention provides in another aspect for an enzyme system comprising a first enzyme and a second enzyme, in which the first enzyme is an enzyme capable of removing free thiols from a proteinaceous substrate, and the second enzyme is a tyrosinase.

The invention provides further for the use of a first enzyme for reducing the concentration of free thiols in a proteinaceous substrate, such as a sulfhydryl oxidase.

The invention provides in another aspect for a method for improving the enzymatic activity on a proteinaceous substrate comprising free thiols by a second enzyme, the activity of which is inhibited by the presence of free thiols, said method comprising treatment of the proteinaceous substrate with a first enzyme capable of removing free thiols; wherein the treatment with the first enzyme is performed prior to, or simultaneous with a treatment with the second enzyme and wherein the first enzyme and the second enzyme is not the same enzyme.

In a further aspect, the invention provides for a method for the preparation of a cross-linked proteinaceous product, said method comprising the steps:

a. treatment of a proteinaceous substrate with a first enzyme capable of degrading or oxidising free thiols;

b. treatment of the proteinaceous substrate with a second enzyme;

wherein step a) is performed prior to, or simultaneous to step b), and wherein step b) results in the cross-linking of proteins present in said proteinaceous substrate.

The above method results in the preparation of a protein-cross linked proteinaceous product.

The proteinaceous substrate may be a proteinaceous foodstuff.

The proteinaceous product may be a proteinaceous protein cross-linked foodstuff.

The invention also provides for the cross linked proteinaceous product prepared by the above method, and, in the case where the proteinaceous substrate is a proteinaceous foodstuff, the invention also provides for food products which comprise the proteinaceous protein cross linked foodstuff product.

The invention also provides for a proteinaceous foodstuff or food product comprising myosin protein, which exhibits an enhanced water holding capacity and/or an increased gel strength, and in which at least a proportion of the myosin protein present in the proteinaceous food product is cross-linked. Water holding capacity and gel strength of the product may be compared to similar products made without use of the method of the invention, or as otherwise defined herein.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings:

FIG. 1: Meat extracts treated with TrTyr2 and TGase. The lanes contain the following extract/treatment: 1) turkey/TGase, 2) turkey/TrTyr2, 3) turkey/reference, 4) cod/TGase, 5) cod/TrTyr2, 6) cod/reference, 7) beef/TGase, 8) beef/TrTyr2, 9) beef/reference, 10) pork/TGase, 11) pork/TrTyr2, 12) pork/reference 13) chicken/TGase, 14)chicken/TrTyr2, 15) chicken/reference, 16) lamb/TGase, 17) lamb/TrTyr2, 18) lamb/reference.

FIG. 2: Pork extracts dialysed against buffers with (lanes 1-3) and without (lanes 4-6) STPP. Extracts are treated with TGase (lanes 1 and 4), with TrTyr2 (lanes 2 and 5) or are references (lanes 3 and 6).

FIG. 3: SDS-PAGE gels of extracts treated with sulfhydryl oxidase before further enzyme treatment. “High SOX” stands for a treatment with 0.33U/ml. Low SOX stands for 0.033 nkat/ml. a) extracts from pork. Lanes contain (first enzyme treatment/second enzyme treatment): 1) high sulfhydryl oxidase/TGase, 2) high sulfhydryl oxidase/TrTyr2, 3) high sulfhydryl oxidase/no treatment, 4) low sulfhydryl oxidase/TGase, 5) low sulfhydryl oxidase/TrTyr2, 6) low sulfhydryl oxidase/no treatment, 7) no treatment/TGase, 8) no treatment/TrTyr2, 9) no treatment/no treatment. b) extracts from beef. Lanes contain (first enzyme treatment/second enzyme treatment): 1) high sulfhydryl oxidase/TGase, 2) high sulfhydryl oxidase/TrTyr2, 3) high sulfhydryl oxidase/no treatment, 4) low sulfhydryl oxidase/TGase, 5) low sulfhydryl oxidase/TrTyr2, 6) low sulfhydryl oxidase/no treatment, 7) no treatment/TGase, 8) no treatment/TrTyr2, 9) no treatment/no treatment.

FIG. 4 illustrates that both gel-strength and WHC of gels made from the different meat extracts are positively correlated with protein content. Note that this figure only depicts reference samples (no enzyme treatment). The gel made from cod extract was inhomogeneous, which gives large variations in gel strength measurement. However, it was consistently lower than for the other gels.

FIG. 5: a: gel-strengths of different heat induced gels produced from protein extracts, which were treated with either water (Ref; n=3), Tyrosinase alone (TrTyr2; n=2), tyrosinase and sulfhydryl oxidase simultaneously (TrTyr2+sulfhydryl oxidase; n=1) or transglutaminase (TGase n=2). b: Similar to a but depicting WHC. Error bars represent the standard deviation.

### DETAILED DESCRIPTION

In one aspect a method is disclosed for the preparation of a cross-linked proteinaceous substrate, said method comprising the steps:

a. treatment of a proteinaceous substrate comprising free thiols with a first ° enzyme capable of removing free thiols;

b. treatment of the proteinaceous substrate with a second enzyme, the activity of which is inhibited by free thiols;

wherein step a) is performed prior to, or simultaneous to step b), and wherein step b) results in the cross-linking of proteins present in said proteinaceous substrate and wherein the first enzyme and the second enzyme is not the same enzyme.

In a further aspect, a method is disclosed for improving the enzymatic activity on a proteinaceous substrate comprising free thiols by a second enzyme, the activity of which is inhibited by the presence of free thiols, said method comprising treatment of the proteinaceous substrate with a first enzyme capable of removing free thiols; wherein the treatment with the first enzyme is performed prior to, or simultaneous with a treatment with the second enzyme and wherein the first enzyme and the second enzyme is not the same enzyme.

The present inventors have found that the inhibition of tyrosinase enzymes in proteinaceous substrates may be due to the presence of free thiols present in the proteinaceous substrate. The present invention is based upon the removal of at least part of the free thiol content of the proteinaceous substrate, thereby alleviating, at least partially, the inhibition of the second enzyme such as tyrosinase.

The invention is particularly relevant to the meat processing industry, where tyrosinases are considered as suitable processing aids for increasing water retention, improving gel strength and improving texture and mouth-feel of processed meat products.

The invention provides for a method for the preparation of a cross-linked proteinaceous substrate, said method comprising the steps:

a. treatment of a proteinaceous substrate comprising free thiols with a first enzyme capable of removing free thiols;

b. treatment of the proteinaceous substrate with a second enzyme, the activity of which is inhibited by free thiols;

wherein step a) is performed prior to, or simultaneous to step b), and wherein step b) results in the cross-linking of proteins present in said proteinaceous substrate and wherein the first enzyme and the second enzyme is not the same enzyme.

The invention further provides for a method for the preparation of a cross-linked protein product, such as protein cross-linked foodstuff, said method comprising the steps:
a. treatment of a proteinaceous substrate with a first enzyme capable of degrading or oxidising free thiols;
b. treatment of the proteinaceous substrate with a second enzyme;

wherein step a) is performed prior to, or simultaneous to step b), and wherein step b) results in the cross-linking of proteins present in said proteinaceous substrate to produce the cross-linked protein product.

The invention further provides for a method for improving the enzymatic activity on a proteinaceous substrate comprising free thiols by a second enzyme, the activity of which is inhibited by the presence of free thiols, said method comprising treatment of the proteinaceous substrate with a first enzyme capable of removing free thiols; wherein the treatment with the first enzyme is performed prior to, or simultaneous with a treatment with the second enzyme and wherein the first enzyme and the second enzyme is not the same enzyme. In one embodiment, the treatment with the first enzyme such as in step a) results in a reduction in the free thiol concentration of the proteinaceous substrate of at least 5%.

In one embodiment, the treatment with the first enzyme such as in step a) results in a reduction in the free thiol concentration of the proteinaceous substrate by at least 1 μM.

In one embodiment, the treatment with the first enzyme such as in step a) results in a reduction in free thiol concentration of the proteinaceous substrate which is sufficient to enhance the ability of the second enzyme to cross link the protein, which may be myosin, present in the substrate, such as a proteinaceous foodstuff.

Cross-linking of protein present in the cross-linked proteinaceous product may be determined by SDS-PAGE analysis of i) the proteins present in the proteinaceous substrate as compared to ii) the cross-linked proteinaceous product of the invention obtained by the method of the invention. An increase in the average size of proteins in the SDS-PAGE in ii) as compared to i) is indicative of a protein cross-linked product. Reduced SDS-PAGE is appropriate as the protein cross-links formed by the activity of tyrosinase are not affected by reducing agents such as dithiothreitol or mercaptoethanol.

In one embodiment, the proteinaceous foodstuff comprises myosin, and the degree of cross-linking of the myosin protein present in the treated (i.e. cross-linked) proteinaceous food products, subsequent to steps a) and b) is at least 5%.

The above method can be used for enhancing the gel strength or water holding capacity, or both, of the proteinaceous substrate (e.g. foodstuff)—so that after steps a) and b) are performed one or more of these parameters are enhanced in the protein cross linked proteinaceous product as compared to the proteinaceous substrate.

The first enzyme is typically selected as an enzyme which is capable of alleviating the inhibition of the second enzyme within a proteinaceous substrate, such as a meat extract, for example a pork meat extract. The examples provide protocols of how such meat extracts may be performed. The tyrosinase spot assay, as disclosed in the examples, may be used to measure tyrosinase inhibition, and alleviation thereof, using either free cysteine as a control inhibitor, or a meat extract, such as a pork meat extract as prepared according to the examples.

From the present analysis, it is considered that one of, or the key inhibitor species present in proteinaceous substrates is free thiol, such as free cysteine. Therefore, in such an embodiment, the enzyme, which is capable of removing free thiols from a proteinaceous substrate, is referred to as the ‘first enzyme’ herein.

It should be recognised that the term ‘removing free thiols’ refers to the decrease (i.e. reduction) in concentration of free thiols present in the proteinaceous substrate, which may, suitably, be performed using a first enzyme, which may be an oxidase.

It should therefore be considered that the term ‘removing free thiols’ does not relate, necessarily, to the preparation of proteinaceous substrates/products which lack any free thiols, just that the concentration of free thiols is reduced.

In one embodiment, the term ‘removing free thiols’ or ‘reducing the concentration of free thiols’ refers to the ‘oxidation of free thiols’.

Therefore, the invention provides for the use of the first enzyme, for reducing the concentration of free thiols in a proteinaceous substrate, such as a proteinaceous foodstuff.

The removal of free thiols from the proteinaceous substrate may be performed using an enzyme, which is capable of removing free thiols from a proteinaceous substrate. Such enzymes may be identified using a free thiol depletion assay. In one embodiment, the free thiol depletion assay is used with a minced pork substrate extraction prepared as according to the Examples.

One unit of enzyme activity is defined as the amount of enzyme that consumes 1 μmol of free thiol groups, such as free cysteine or glutathione, per minute with 1 mM as substrate at pH 7.4 and 30° C. in phosphate buffer.

The catalytic activity may also be defined in katal (SI unit for catalytic activity) which is the amount of enzyme needed to convert 1 mol of substrate pr. second at a pre-defined substrate concentration, temperature and pH.

Thus 1 nkat/g is equivalent to a specific enzyme activity of 0.06 units/g or 1 unit/g is equivalent to 16.6 nkat/g.

One method of removing free thiols from a proteinaceous substrate is to oxidise the free thiol component, or part thereof—therefore in such an embodiment, the first enzyme is preferably an oxidase (oxido-reductase) enzyme.

In one embodiment, the first enzyme is an oxidase enzyme, such as an enzyme which exhibits one or more of the following activities: sulfhydryl oxidase, laccase (E.C. 1.10.3.2), glutathione oxidase, and disulphide isomerase.

In one embodiment, the first enzyme exhibits an enzyme activity selected from the group consisting of: EC 1.8.3.2 and 1.8.3.3.

In one embodiment, the first enzyme is a sulfhydryl oxidase, or exhibits sulfhydryl oxidase activity, i.e. E.C. 1.8.3.2.

In one embodiment, the first enzyme is an enzyme capable of generating hydrogen peroxide, such as a carbohydrate oxidase, such as glucose oxidase or hexose oxidase. In such as embodiment, the inventors consider that the hydrogen peroxide generated oxidises the free thiols present, thereby alleviating the inhibition of the tyrosinase.

The term sulfydryl oxidase (SOX) is defined by the enzyme classification E.C. 1.8.3.2 as set out by the Nomenclature Committee of the International Union of Biochemistry and Molecular Biology (IUBMB) as an enzyme which converts thiol compounds to their corresponding disulfides according to the equation:2 RSH+0₂→RSSR+H₂O₂.

Numerous sulfhydryl oxidases are known in the art, and may be used for the purposes of the present invention. In one embodiment, the sulfhydryl oxidase may be isolated from, or derived from a microbial source, such as a fungus, such as yeast or Aspergillus. EP 0 321 881 EP 0 338 452 and EP 0 705 538 report on the use of microbial sulfydryl oxidases in bakery application.

A preferred sulfydryl oxidase may be derived from Aspergillus niger, such as the sulfydryl oxidase described in de la Motte and Wagner Biochemistry 26 (1987) 7363-737.

U.S. Pat. No. 4,632,905, provides a sulfydryl oxidase from Aspergillus sojae hereby incorporated by reference.

Other sulfydryl oxidases include bovine sulfydryl oxidase.

Aspergillus SOX Enzymes—NCBI Accession Numbers—The following records are hereby incorporated by reference.

1: CAB58940. unnamed protein p . . . [gi:6090366]

2: EAU34803. FAD-linked sulfhy . . . [gi:114193103]

3: XP_{—}754731. FAD dependent sul . . . [gi:71000028]

4: XP_{—}749041. FAD dependent sul . . . [gi:70987121]

5: XP_{—}001213534. FAD-linked sulfhy . . . [gi:115395790]

6: EAL92693. FAD dependent sul . . . [gi:66852368]

7: EAL87003. FAD dependent sul . . . [gi:66846671]

8: XP_{—}001273306. FAD dependent sul . . . [gi:121711381]

9: XP_{—}001270904. FAD dependent sul . . . [gi:121705282]

10: EAW11880. FAD dependent sul . . . [gi:119401457]

11: EAW09478. FAD dependent sul . . . [gi:119399050]

12: CAK40401. sulphydryl oxidas . . . [gi:134078459]

13: XP_{—}001401908. hypothetical prot . . . [gi:145257982]

14: CAK38806. unnamed protein p . . . [gi:134074512]

15: CAL00471. unnamed protein p . . . [gi:134083103]

16: CAK49098. unnamed protein p . . . [gi:134078570]

17: XP_{—}001397560. hypothetical prot . . . [gi:145252094]

18: BAE55270. unnamed protein p . . . [gi:83765127]

19: XP_{—}001402401. hypothetical prot . . . [gi:145256027]

20: XP_{—}001208942. augmenter of live . . . [gi:115384790]

21: EAU38334. augmenter of live . . . [gi:114196634]

22: XP_{—}661363. hypothetical prot . . . [gi:67526603]

23: XP_{—}660631. hypothetical prot . . . [gi:67525139]

24: EAA63598. hypothetical prot . . . [gi:40744422]

25: EAA59967. hypothetical prot . . . [gi:40740777]

Yeast SOX—Enzymes—NCBI Accession Numbers—The following records are hereby incorporated by reference.

1: EDN61625sulfhydryl oxidas . . . [gi:151943312]

2: EDN61173sulfhydryl oxidas . . . [gi:151942827]

3: P27882Mitochondrial FAD . . . [gi:2506175]

4: Q12284FAD-linked sulfhy . . . [gi:2492823]

5: 1JRA_DChain D, Crystal . . . [gi:18158801]

6: 1JRA_CChain C, Crystal . . . [gi:18158800]

7: 1JRA_BChain B, Crystal . . . [gi:18158799]

8: 1JRA_AChain A, Crystal . . . [gi:18158798]

9: 1JR8_BChain B, Crystal . . . [gi:18158797]

10: 1JR8_AChain A, Crystal . . . [gi:18158796]

11: P55789FAD-linked sulfhy . . . [gi:2492821]

12: CAA92143unknown [Saccharo . . . [gi:1072405]

13: AAB48659regulatory protei . . . [gi:172378]

14: CAB16284sulfhydryl oxidas . . . [gi:2408079]

15: P36046Intermembrane spa . . . [gi:90110034]

16: CAB46757sulfhydryl oxidas . . . [gi:5441486]

17: NP_{—}011543Erv1p [Saccharomy . . . [gi:6681846]

18: NP_{—}015362Erv2p [Saccharomy . . . [gi:6325296]

19: CAA97017ERV1 [Saccharomyc . . . [gi:1945314]

20: CAA97016ERV1 [Saccharomyc . . . [gi:1945313]

21: CAA48192ERV1 [Saccharomyc . . . [gi:404218]

22: CAA94987unknown [Saccharo . . . [gi:1314111]

23: CAA43129regulatory protei . . . [gi:4305]

24: CAH56919unnamed protein p . . . [gi:52747921]

25: NP_{—}005685COX17 homolog, cy . . . [gi:5031645]

26: XP_{—}570710thiol oxidase [Cr . . . [gi:58267108]

27: AAW43403thiol oxidase, pu . . . [gi:57226944]

28: CAL00471unnamed protein p . . . [gi:134083103]

29: NP_{—}005253erv1-like growth . . . [gi:54112432]

30: CAK38806unnamed protein p . . . [gi:134074512]

31: XP_{—}001397560hypothetical prot . . . [gi:145252094]

32: AAA96390ERV1 [Homo sapien . . . [gi:6136037]

33: CAD25469PROTEIN OF THE ER . . . [gi:19069001]

34: XP001644445hypothetical prot . . . [gi:156842152]

35: XP_{—}001642797hypothetical prot . . . [gi:156837548]

36: ED016587hypothetical prot . . . [gi:156115088]

37: ED014939hypothetical prot . . . [gi:156113366]

38: XP_{—}001382524predicted protein . . . [gi:150863891]

39: XP_{—}001383309predicted protein . . . [gi:126133569]

40: ABN64495predicted protein . . . [gi:149385147]

41: ABN65280predicted protein . . . [gi:126095458]

42: XP_{—}570522hypothetical prot . . . [gi:58266732]

43: AAW43215conserved hypothe . . . [gi:57226755]

A suitable sulfyhryl oxidase is provided by EP 0 565 172, hereby incorporated by reference, as shown in SEQ ID NO 1

SEQ ID NO 1:
>gi|134078459|emb|CAK40401.1| sulphydryl oxidase
Sox from patent
EP565172-A1-Aspergillus niger
MAPKSLFYSLFSTISVALASSIPQTDYDVIVVGGGPAGLSVLSSLGRMRR
KTVMFDSGEYRNGVTREMHDVLGFDGTPPAQFRGLARQQISKYNSTSVID
IKIDSITPVEDAAANSSYFRAVDANGTQYTSRKVVLGTGLVDVIPDVPGL
REAWGKGIWWCPWCDGYEHRDEPLGILGGLPDVVGSVMETHTLYSDIIAF
TNGTYTPANEVALAAKYPNWKQQLEAWNVGIDNRSIASIERLQDGDDHRD
DTGRQYDIFRVHFTDGSSVVRNTFITNYPTAQRSTLPEELSLVMVDNKID
TTDYTGMRTSLSGVYAVGDCNSDGSTNVPHAMFSGKRAGVYVHVEMSREE
SNAAISKRDFDRRALEKQTERMVGNEMEDLWKRVLENHHRRS

Laccase (EC 1.10.3.2) has been reported to oxidize SH groups directly (Ref: Applied and Environmental Microbiology, February 2000, p. 524-528, Vol. 66, No. 2). Therefore, in one embodiment, the first enxyme is a laccase, or exhibits laccase activity.

The term “laccase activity” is defined by the enzyme classification EC 1.10.3.2 (laccase) as set out by the Nomenclature Committee of the International Union of Biochemistry and Molecular Biology (IUBMB); or enzyme classification EC 1.10.3.1 (catechol oxidase), enzyme classification EC 1.10.3.4 (o-aminophenol oxidase), or enzyme classification EC 1.3.3.5 (bilirubin oxidase).

Numerous laccase enzymes are known in the art, for example those disclosed in: WO2007/054034A—which reports on a laccase from Streptomyces coelicolor, particularly the laccase represented by 1 to 343 of SEQ ID NO:2 of WO2007/054034A, hereby incorporated by reference. In one embodiment, of the invention the oxidase is a laccase, such as a Polyporus sp. laccase such as the Polyporus pinisitus laccase (also called Trametes villosa laccase) described in WO 96/00290 (from Novo Nordisk Biotech., inc.) or a Myceliophthera sp. laccase especially the Myceliophthera thermophila laccase described in WO 95/33836 (from Novo Nordisk Biotech inc.). Further, the laccase may be a Scytalidium sp. laccase, such as the S. thermophilium laccase described in WO 95/33837 (from Novo Nordisk Biotech inc.) or a Pyricularia sp. laccase, such as the Pyricularia oryzae laccase which can be purchased from SIGMA under the trade name SIGMA no. L5510, or a Coprinus sp. laccase, such as a C. cinereus laccase, especially a C. cinereus IFO 30116 laccase, or a Rhizoctonia sp. laccase, such as a Rh. solani laccase, especially the neutral Rh. solani laccase described WO 95/07988 (from Novo Nordisk A/S) having a pH optimum in the range from 6.0 to 8.5. The laccase may also be derived from a fungi such as Collybia, Fomes, Lentinus, Pleurotus, Aspergillus, Neurospora, Podospora, Phlebia, e. g. P. radiata (WO 92/01046), Coriolus sp., e. g. C. hirsitus (JP 2-238885), or Botrytis. Further laccases are disclosed in WO01/83770.

The term “glutathione oxidase” (GOX) is defined as enzyme classification (E.C. 1.8.3.3) as set out by the Nomenclature Committee of the International Union of Biochemistry and Molecular Biology (IUBMB) as an enzyme which catalyses the reaction 2 glutathione+O(2)=oxidized glutathione+H(2)O(2), and may also catalyse the oxidation of cysteine and several other thiols. In one embodiment, glutathione activity may be determined by measuring the disappearance of glutathione in buffer solution in the presence of oxygen. In such an embodiment, one ‘GOX’ unit is the amount of enzyme needed to oxidize 1 μmole glutathione per minute at 30° C. and pH 7.

In one embodiment, the first enzyme has a specific activity of at least 0.01 nkat/mg, such as at least 0.1 nkat/mg, such as at least 1 nkat/mg, such as at least 10 nkat/mg, such as at least 50 nkat/mg, such as at least 100 nkat/mg, such as at least 200 nkat/mg, such as at least 300 nkat/mg. In one embodiment, the first enzyme has a specific activity of no greater than 500 nkat/mg, such as no greater than 300 nkat/mg, such as no greater than 100 nkat/mg, such as no greater than 10 nkat/mg, such as no greater than 5 nkat/mg, such as no greater than 1 nkat/mg.

In one embodiment, the first enzyme is added to the proteinaceous substrate, in an amount of between 0.00001 mg/g and 100 mg/g of proteinaceous substrate, such as between 0.01 mg/g and 10 mg/g, such as between 0.10 mg/g and 5 mg/g. In one embodiment, at least 0.00001 mg of the first enzyme is added per g of proteinaceous substrate, such as at least 0.0001 mg/g, such as at least 0.001 mg/g, such as at least 0.01 mg/g. In one embodiment, up to 10 mg of the first enzyme is added per g of proteinaceous substrate, such as up to 5 mg/g, such as up to 1 mg/g, such as up to 0.5 mg/g, such as up to 0.2 mg/g.

In one embodiment, the amount of first enzyme added is between 0.01 and 1 nkat/g of proteinaceous substrate (or foodstuff), such as between 0.02 and 0.5 nkat/g.

The removal of the inhibitory compounds in the proteinaceous substrate may allow for a more effective action of the second enzyme, such as a more effective protein cross-linking of the protein present in the substrate e.g. by tyrosinase.

In one embodiment, the second enzyme is an enzyme which catalytic action is inhibited by the presence of free thiols. In one embodiment, the catalytic action of the second enzyme such as tyrosinase in a proteinaceous substrate will result in cross-linking of proteins.

In one embodiment, the term “improvement” is defined by an increase in the catalytic activity of the second enzyme in the product treated according to the invention as compared to the catalytic activity of the second enzyme in the product without treatment with the first enzyme.

In one embodiment, the term “improvement” is defined by a change in a measurable parameter (such as gel strength), which will be perceived as an improvement by the manufacturer or consumer.

In one embodiment, the term “improvement” is defined by an improvement in water holding capacity in the product treated according to the invention as compared to the water holding capacity in the product without treatment with the first enzyme.

In another embodiment, the term “improvement” is defined by an improvement in cross-linking in the product treated according to the invention as compared to the cross-linking in the product without treatment with the first enzyme.

In another embodiment, the term “improvement” is defined by an improvement in the texture of the product treated according to the invention as compared to the texture of the product without treatment with the first enzyme.

In one embodiment, the enzymatic activity results in the cross-linking of proteins present in a proteinaceous substrate such as meat. An example of a second enzyme which is suitable for use for cross-linking of proteins in a proteinaceous substrate such as meat is tyrosinase.

In one embodiment, the first enzyme and the second enzyme is not the same enzyme.

In one embodiment, the second enzyme is selected from the group consisting of a tyrosinase, laccase, lipoxygenase, galactose oxidase, protein lysin 6-oxidase (lysyl oxidase), galactolipase and lysophospholipase,

In one embodiment, the second enzyme is selected from the group consisting of a tyrosinase, laccase, lipoxygenase and protein lysin 6-oxidase (lysyl oxidase), In a further embodiment, the second enzyme is selected from the group consisting of lipoxygenase and protein lysin 6-oxidase (lysyl oxidase). In a further embodiment, the second enzyme is laccase. Most preferably the enzyme is a tyrosinase belonging to the group described by the EC number: 1.14.18.1.

In one aspect, the second enzyme is tyrosinase, which is defined by the enzyme classification EC E.C. 1.14.18.1 as set out by the Nomenclature Committee of the International Union of Biochemistry and Molecular Biology (IUBMB) (also referred to a monophenyl monoxoygenase).

Tyrosinase belongs to the group of phenol oxidases, which use oxygen as electron acceptor. Traditionally tyrosinases can be distinguished from other phenol oxidases, i.e. laccases, on the basis of substrate specificity and sensitivity to inhibitors. However, the differentiation is nowadays based on structural features. Structurally the major difference between tyrosinases and laccases is that tyrosinase has a binuclear copper site with two type III coppers in its active site, while laccase has altogether four copper atoms (type I and M coppers, and a pair of type III coppers) in the active site. Tyrosinase oxidizes various phenolic compounds to the corresponding quinones. The quinones are highly reactive and may react further non-enzymatically. A typical substrate of tyrosinase is tyrosine (or tyrosine residue in proteins), which is first hydroxylated into DOPA (dihydroxyphenylalanine or DOPA residue in proteins)), which is then further oxidized by the enzyme to dopaquinone (or dopaquinone residue in proteins). Dopaquinone may react non-enzymatically with a number of chemical structures, such as other dopaquinones, thiol and amino groups. Tyrosinase thus has two enzyme activities in one and the same protein, i.e. monophenol monooxyganase activity (EC 1.14.18.1) and catechol oxidase activity (EC 1.10.3.1) as shown below.

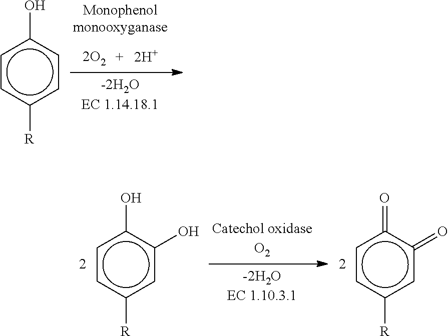

The substrate specificity of tyrosinase is relatively broad, and the enzyme is capable of oxidizing a number of polyphenols and aromatic amines. Contrary to laccase (EC 1.10.3.2), however, tyrosinase does not oxidize syringaldazin. At least tyrosine, lysine and cysteine residues in proteins form covalent bonds with active dopaquinones catalysed by tyrosinase.

Tyrosinase activity can be measured by techniques generally known in the art. L-DOPA or L-tyrosine can be used as a substrate, whereafter dopachrome formation may be monitored spectrofotometrically, or alternatively substrate consumption may be monitored by following the oxygen consumption.

Tyrosinases are widely distributed in nature, and they are found in animals, plants, fungi and bacteria. Especially vegetables and fruits susceptible of browning are rich in tyrosinase. The only commercially available tyrosinase at present is derived from the mushroom Agaricus bisporus. The tyrosinase used in the present invention may originate from any animal, plant, fungus or microbe capable of producing tyrosinase. According to one embodiment of the invention, the tyrosinase is derived from a filamentous fungus. It may for example be an extracellular tyrosinase obtainable from Trichoderma reesei (WO 2006/084953) hereby incorporated by reference.

Tyrosinases catalyse the reaction L-tyrosine+L-dopa+O(2)→L-dopa+dopaquinone+H(2)O.

In one embodiment, the tyrosinase is isolated or derived from a fungal species, such as Trichoderma or Hypocrea.

Selinheimo et al., FEBS Lett. 273, 4322-4335 (2006), hereby incorporated by reference, provides a tyrosinase from Trichoderma.

In one embodiment, the tyrosinase is as disclosed in SEQ ID NO 2, taking into account co- and post-translational modifications, such as signal peptide cleavage:

SEQ ID NO 2
>gi|118764455|emb|CAL90884.1| tyrosinase 2
[Hypocrea jecorina]
MLLSASLSALALATVSLAQGTTHIPVTGVPVSPGAAVPLRQNINDLAKSG
PQWDLYVQAMYNMSKMDSHDPYSFFQIAGIHGAPYIEYNKAGAKSGDGWL
GYCPHGEDLFISWHRPYVLLFEQALVSVAKGIANSYPPSVRAKYQAAAAS
LRAPYWDWAADSSVPAVTVPQTLKINVPSGSSTKTVDYTNPLKTYYFPRM
SLTGSYGEFTGGGNDHTVRCAASKQSYPATANSNLAARPYKSWIYDVLTN
SQNFADFASTSGPGINVEQIHNAIHWDGACGSQFLAPDYSGFDPLFFMHH
AQVDRMWAFWEAIMPSSPLFTASYKGQSRFNSKSGSTITPDSPLQPFYQA
NGKFHTSNTVKSIQGMGYSYQGIEYWQKSQAQIKSSVTTIINQLYGPNSG
KKRNAPRDFLSDIVTDVENLIKTRYFAKISVNVTEVTVRPAEINVYVGGQ
KAGSLIVMKLPAEGTVNGGFTIDNPMQSILHGGLRNAVQAFTEDIEVEIL
SKDGQAIPLETVPSLSIDLEVANVTLPSALDQLPKYGQRSRHRAKAAQRG
HRFAVPHIPP
L

The full sequence and the details of the signal peptide processing are described in WO06/084953, hereby incorporated by reference.

One unit of tyrosinase activity—1 nkat is defined as the amount of enzyme that converts 1 nmol of L-DOPA into DOPA-quinone pr. second at pH 7 and 25° C.

In one embodiment, the second enzyme is a laccase as defined above.

In one embodiment, the second enzyme is a lipoxygenase defined by the enzyme classification EC 1.13.11. Lipoxygenases are a class of iron-containing dioxygenases, which catalyzes the hydroperoxidation of lipids, containing a cis,cis-1,4-pentadiene structure. The primary products are hydroperoxy fatty acids, which usually are rapidly reduced to hydroxy derivatives.

In one embodiment, the second enzyme is galactose oxidase. A galactose oxidase enzyme catalyzes the stereospecific oxidation of primary alcohols to the corresponding aldehydes. The biologically most relevant substrate of the enzyme is not known as the enzyme exhibits broad substrate specificity. In one embodiment, galactose is a substrate.

In one embodiment, the second enzyme is a lysin 6-oxidase (lysyl oxidase) defined by the enzyme classification EC 1.4.3.13. A lysyl oxidase is an extracellular copper-dependent enzyme that catalyzes the oxidative deamination of peptidyl lysine residues in precursors of various collagens and elastins. The deaminated lysines are then able to form aldehyde cross-links.

In one embodiment, the second enzyme is a galactolipase defined by the enzyme classification E.C. 3.1.1.26. A galactolipase is capable of hydrolysing at least 1,2-diacyl-3-beta-D-galactosyl-sn-glycerol. Generally galactolipases are also able to hydrolyse 2,3-di-O-acyl-1-O-(6-O-α-D-galactosyl-β-D-galactosyl)-D-glycerol, and phosphatidylcholine and other phospholipids. However, they are incapable, or substantially incapable, of hydrolysing a triglyceride and/or a 1-monoglyceride.

In one embodiment, the second enzyme is a lysophospholipase defined by the enzyme classification EC 3.1.1.5. A lysophospholipase is an enzyme that catalyzes the hydrolysis of a single fatty acid ester bond in lysoglycerophosphatidates with the formation of glyceryl phosphatidates and a fatty acid.

Second Enzyme Dosages.

The dosage of the second enzyme used such as tyrosinase may be, for example at least 20, at least 40, at least 80, at least 160, at least 320 or at least 640 nkat/g of protein. Applicants find that an amount between 100 and 500, such as between 200-200, or 250-350 or (about) 300 nkat/g of meat protein is usually suitable for cross-linking of meat proteins.

In one embodiment, the second enzyme such as tyrosinase has a specific activity of at least 0.1 nkat/mg, such as at least 1 nkat/mg, such as at least 10 nkat/mg, such as at least 100 nkat/mg, such as at least 200 nkat/mg, such as at least 250 nkat/mg, such as at least 300 nkat/mg.

In one embodiment, the second enzyme such as tyrosinase has a specific activity of no greater than 1000 nkat/mg, such as no greater than 800 nkat/mg, such as no greater than 500 nkat/mg. The enzyme used in the examples (derived from SEQ ID NO 2) has a specific activity of about 300 nkat/mg.

In one embodiment, the second enzyme is added to the proteinaceous substrate, in an amount of between 0.00001 mg/g and 100 mg/g of proteinaceous substrate, such as between 0.01 mg/g and 10 mg/g, such as between 0.10 mg/g and 5 mg/g. In one embodiment, at least 0.00001 mg of the second enzyme is added per g of proteinaceous substrate, such as at least 0.0001 mg/g, such as at least 0.001 mg/g, such as at least 0.01 mg/g. In one embodiment, up to 10 mg of the second enzyme is added per g of proteinaceous substrate, such as up to 5 mg/g, such as up to 1 mg/g, such as up to 0.5 mg/g, such as up to 0.2 mg/g.

In one embodiment, the amount of second enzyme added is between 0.001 and 10 nkat/ml, such as between 0.01 nkat/ml and 1 nkat/ml, of proteinaceous substrate (or foodstuff), such as between 0.02 and 0.5 nkat/ml.

Enzymes and their Preparation

The first and second enzymes may be isolated from their natural source or prepared by synthetic or recombinant techniques.

With regards the amino acid sequence of the enzymes referred to herein, and referring ‘to the sequences disclosed or referenced herein, it is recognised that the sequences may be variants, homologues or fragments—these terms are used as defined in EP 1 704 2408. Suitably the enzyme may have a homology of at least 70%, such as at least 80%, such as at least 90%, such as at least 95%, such as at least 98% homologous or 100% homologous to the (mature) sequences as disclosed or referenced herein.

Typically, the expression of the first and or second enzyme is a eukaryotic host, such as native of a foreign host cell, results in the co and post-translational modifications, such as signal peptide cleavage.

EP 1 704 240B provides a description of the standard recombinant technology which can be employed for the expression and preparation of enzymes.

Free Thiols

Thiols contain the —SH group, which also known as the thiol group. A free thiol, is a thiol containing compound, such as an amino acid, or a short peptide with a molecular weight of less than 1000 g/mol, such as less than 500 g/mol, such as less than 350 g/mol, such as less than 200 g/mol.

Free thiols, which may typically be present or formed in a proteinaceous substrate include, cysteine, glutathione, preferably cysteine.

In one embodiment, the free thiol is a —SH group containing (free) amino acid, such as free cysteine—i.e. cysteine which is present as an amino acid monomer, and not part of a polypeptide chain.

In one embodiment, the ‘free thiol’ is a peptide, comprising one or more thiol groups, wherein the peptide consists of between 2-8 amino acids residues in lengths, such as a peptide consisting of 2, 3, 4, 5, 6, 7 or 8 amino acids, such as di- or tri-peptides, such as glutathione. Suitably, in a preferred embodiment the peptide comprises one or more cysteine residues.

The presence or concentration of free thiols present in a proteinaceous substrate may be determined by the free thiol assay provided in the examples—please note that the protocol for the preparation of a meat extract can also be used with other proteinaceous substrates.

In one embodiment, the free thiol concentration in the proteinaceous (food) substrate, which may be treated with the first enzyme, has a free thiol concentration, prior to treatment with the first enzyme, of at least 2 μM, such as at least 3 μM, such as at least 4 μM, such as at least 5 μM, such as at least 6 μM, such as at least 7 μM, such as at least 8 μM, such as at least 9 μM, such as at least 10 μM, such as at least 11 μM, such as at least 12 μM, such as at least 13 μM, such as at least 14 μM, such as at least 15 μM, such as at least 16 μM, such as at least 17 μM.

In one embodiment, the free thiol concentration in the proteinaceous (food) substrate, which may be treated with the first enzyme, has a free thiol concentration, prior to treatment with the first enzyme of between 5-25 μM, such as between 10-25 μM, such as between 15-20 μM.

In one embodiment, the free thiol concentration of the proteinaceous (food) substrate is reduced by at least 5%, such as at least 10%, such as at least 20%, such as at least 30%, such as at least 40%, such as at least 50%, such as at least 60%, such as at least 70%, such as at least 80%, by the treatment with the first enzyme (such as after step a)).

In one embodiment, the free thiol concentration of the proteinaceous (food) substrate after the treatment with the first enzyme (such as after step a)), is less than 15 μM, such as less than 10 μM, such as less than 9 μM, such as less than 8 μM, such as less than 7 μM, such as less than 6 μM, such as less than 5 μM, such as less than 4 μM, such as less than 3 μM.

In one embodiment, the reduction in the free thiol concentration of the proteinaceous food substrate is (such as after step a) at least 1 μM, such as at least 2 μM, at least 3 μM, at least 4 μM, at least 5 μM, at least 6 μM, at least 7 μM, at least 8 μM, at least 9 μM, at least 10 μM, at least 11 μM, at least 12 μM, at least 13 μM, at least 14 μM, at least 15 μM, such as at least 16 μM.

In one embodiment, the reduction in free thiol concentration of the proteinaceous food substrate is sufficient to enhance the ability of a second enzyme to cross link the protein present in the food substrate, such as myosin.

A proteinaceous food substrate may be in the form of a foodstuff or food product, preferably a foodstuff. Preferably, the proteinaceous (food) substrate comprises meat or meat protein.

In a further embodiment, the proteinaceous substrate is flour, dough, a cereal derived product, milk, a milk derived product, a dairy product, soy protein, soy milk, or a vegetable derived product.

The proteinaceous substrate is, in one embodiment a foodstuff.

In one embodiment, the proteinaceous substrate comprises or consists of animal meat.

In one embodiment, the proteinaceous substrate, such as the meat substrate, comprises myosin.

The term “proteinaceous” as used herein, refers to a substrate or composition which has a protein content of at least 1%, such as at least 2%, such as at least 3%, such as at least 4%, such as at least 5%, such as at least 10% by weight, such as at least 10% by weight, such as at least 20% by weight, such as at least 30% by weight, such as at least 40% by weight, such as at least 50% by weight.

In a preferred embodiment, the proteinaceous substrate or composition comprises the protein myosin, such as at least 1%, at least 5%, at least 10%, at least 20%, at least 30%, at least 40%, at least 50% of the protein present in the proteinaceous substrate or composition is myosin.

The term myosin as used herein refers to the motor protein found in eukaryotic muscle, i.e. the major protein found in meat. Myosin is identified as a 188 kDa protein by SDS-PAGE analysis. The proportion of myosin protein can therefore be determined by SDS-PAGE analysis.

The term “meat” refers to the flesh of animals which is used as food, preferably comprising muscle flesh optionally with associated fat and connective tissues. Meat as used herein includes any kind of meat of livestock, game, poultry, fish and other edible sea animals. The meat may be e.g. pork, beef, mutton, kangaroo, chicken, turkey, ostrich, fish, molluscs, shark and shellfish etc. “Meat product” refers to any material comprising meat or meat protein as an essential ingredient, such as sausages, hams, restructured meat products, surimi, etc.

The proteinaceous substrate may, in one embodiment be a meat substrate.

In one embodiment, the proteinaceous food substrate contains or is derived from animal meat, such as mammalian, bird, or fish.

In one embodiment, the proteinaceous food source is obtained from a mammal (commonly referred to as red meat), such as pork, lamb, beef, goat, horse, kangaroo etc.

In one embodiment, the proteinaceous food substrate is obtained from a bird, such as poultry, such as chicken, turkey, ostrich.

In one embodiment, the proteinaceous food substrate contains or is derived from fish, such as white fish, cod, haddock, coley etc, cartilaginous fish such as shark, swordfish, sea water fish, tuna, mackerel, sardines, anchovies, herring, salmon, freshwater fish, trout, etc.

In one embodiment, the proteinaceous food substrate contains or is, or comprises, or is derived from meat selected from the group consisting of pork, lamb, chicken, beef, turkey, cod, kangaroo, ostrich, shark.

In one embodiment, the proteinaceous food substrate is, or comprises, or is derived from pork, lamb, and chicken, most preferably pork.

The term pork refers to meat obtained from the carcass of a pig. The term beef refers to meat obtained from the carcass of a cow (male of female bovine). The term lamb refers to meat obtained from a sheep.

Conveniently the meat substrate (or product) may contain at least 20%, at least 30%, at least 40% or at least 45 wt-% (wet) meat. The meat substrate (or product) may contain up to 100% wt-% meat (wet), such as up to 50%, up to 60%, up to 70%, up to 80% and up to 90% meat. For example, 1 restructured meat substrate may in practice comprise up to 100 wt-% meat.

The proteinaceous substrate typically comprises water, such as between 1 wt % and 95wt % water. Meat. (animal muscle) typically comprises about 60-75% water, although meat from different sources and different species may contain differing amounts of water.

In one embodiment, exogenous water is added to the proteinaceous substrate, for example water is added prior to or during steps a) and/ or b) of the method of the invention. In the context of a meat substrate, exogenous water, is, for example water which is not naturally present in or derived from the meat obtained from the animal. In one embodiment, the amount of exogenous water is between 1% and 90% of the original weight of the proteinaceous substrate (i.e. prior to addition of exogenous water), such as between 10 and 80%. The amount of exogenous or added water may be up to 90%, up to 80%, up to 70%, up to 60%, up to 50%, up to 40%, up to 30%, up to 20%, up to 10%. The amount of added water may be at least 1%, such as at least 5%, such as at least 10%, such as at least 20%, such as at least 30%, such as at least 40%, such as at least 50%.

The exogenous water added to the substrate prior to or during the steps a) and/or b) may comprise other ingredients.

It is recognised that exogenous water may become incorporated into the proteinaceous substrate (such as meat substrate) by the method of the invention, to produce a product (such as a meat product) which has enhanced water content as compared to the proteinaceous substrate. Excess water which has not been incorporated into the product may be separated and removed.

Therefore, it is recognised that the method according to the invention may be used to enhance the water holding capacity of a meat product. In this regards, in one preferred embodiment, the water content of the proteinaceous substrate is increased by the enzymatic treatment according to the present invention. For example the water content of the meat product obtained from the method according to the invention, is higher that the water content of the meat substrate prior to enzymatic treatment. Suitably the increase in water content, may, in one embodiment be at least 1%, such as at least 5%, such as at least 10such as at least 15%, such as at least 20%.

The particle size of the comminuted meat (substrate) depends on the type of meat product to be prepared. For the manufacture of restructured meat products, the meat is cut into recognizable pieces with edges of usually several cm, whereas the meat in hams and sausages is usually ground, chopped and/or minced or otherwise homogenized. Typically ham contains coarsely ground meat with particles of several mm up to one or a few cm, whereas sausages contain finely ground meat.

The meat protein (i.e. present in the meat substrate) may be in the form of mechanically separated or recovered meat, and/or meat which has been mechanically disrupted, e.g. by homogenised, mincing, grinding, dicing, liquidising etc (collectively referred to as “comminuting”). In one embodiment, the proteinaceous food substrate is in the form of a meat slurry, reconstituted meat or emulsified meat.

In one embodiment, the meat substrate contains from about 1 to about 30 percent by weight (“% w/w”) animal protein, based on the dry weight of such protein.

In one embodiment, the meat substrate comprises, based on about 100% w/w of the food product, from about 1% w/w to about 30% w/w muscle protein (myosin), from about 30% w/w to about 80% w/w water, up to about 50% w/w fat.

In one embodiment, the proteinaceous substrate, such as meat substrate, is obtained by comminuting meat, optionally in the presence of water.

Meat slurry is typically prepared by a process where the meat is first finely ground and mixed with water. The mixture is then used in a centrifuge or with an emulsifier to separate the fats and myoglobin from the muscle. The product is then allowed to settle into three layers: meat, excess water, and fat. The meat layer is then separated off and either used directly as a foodstuff in the preparation of food products, such as chicken nuggets, sausage, burgers, reconstituted meats etc, or stored for future use (e.g. by freezing).

Mechanically separated meat (MSM), also known as mechanically recovered meat (MRM) is a paste-like and batter-like meat product produced by forcing animal (e.g. beef, pork or chicken), bones, with attached edible meat, under high pressure through a sieve or similar device to separate the bone from the edible meat tissue. Since the advent of BSE disease, the use of mechanically separated meat is less favoured, and recovered meat is preferred.

Recovered meat refers to the residual meat trimmings which are extracted from bones and other carcass materials, without mixing the bone and meat tissues. This meat is comparable in appearance, texture, and composition to meat trimmings and similar meat products derived by hand. The meat is separated from bone by scraping, shaving, or pressing the meat from the bone without breaking or grinding the bone.

The “meat-substrate” comprises meat. In addition, it may, comprise “other ingredients” which encompass any conventional additives, such as NaCI, phosphates, and/or water. Further, the term other ingredients includes e.g. salts other than NaCI and phosphates, spices, preservatives, antioxidants, stabilizers, sugar, sweeteners, gums, binders, extenders, starch, dextrin-type of carbohydrates, animal or vegetable fats and oils, fat substitutes and/or other non-meat ingredients such as soy, casein, and whey, wheat proteins and other non-meat proteins etc.

In one embodiment, the “meat-substrate” consists or comprises of meat and water. The meat substrate may be communited meat.

The term “meat product” refers to the product prepared by the method according to the invention, wherein the proteinaceous substrate is a “meat substrate”. The proteinaceous protein-cross linked proteinaceous product may therefore be a meat product.

In the context of the present invention the product prepared by the method of the invention may be a meat product prepared by binding communited meat by the activity of a second enzyme (tyrosinase). Such meat products may be referred to as restructured (reconstituted). The communited meat is either before or during tyrosinase treatment, treated with a first enzyme according to the invention.

In one embodiment, the meat product prepared may be a fat-reduced product containing 15-18 wt-% fat, or a low-fat meat product containing up to 10 wt-% fat, or a lean meat product containing up to 5 wt-% fat. Preferably the fat content of the meat product is no more than 18 wt-%, preferably no more than 10 wt-%, and most preferably no more than 5 wt-% or even no more than 3 wt-%.

In one embodiment, the meat product may comprise less than 2.0 wt-% of salt, preferably less than 1.5 wt-%. Meat products comprising no more than 1.2 wt-% salt are generally considered as low-salt products. The meat product of the invention therefore preferably contains no more than 1.2 wt-% salt, and, according to one embodiment of the invention, no more than 1.0 wt-%. “Salt” in this respect refers to sodium chloride (NaCl). In one embodiment, the meat substrate and or product is essentially salt free—i.e. the concentration of phosphate is less than 0.1% wt % salt.

The addition of phosphates has increased during the last years, be-cause phosphates may be used to maintain the structure and water-binding ability of low-salt products. Nowadays industry normally adds 0.2 wt-% phosphate (measured as P₂O₅) to a meat product, which corresponds to 0.34 wt-% trisodiumpyrophosphate. The meat substrate and/or product of the present invention may contain less than 0.2 wt-% phosphate, preferably it contains no more than 0.1 wt-% added phosphate (measured as P₂O₅). Most preferably the meat substrate and/or product is phosphate-free, i.e. no phosphate has been added.

In one embodiment, the meat substrate and or product is essentially phosphate free—i.e. the concentration of phosphate is less than 0.1% wt % phosphate.

The term a “foodstuff” as used herein refers to an edible composition which forms part of the human or animal diet. A foodstuff can be used directly as a food, or used in the preparation of a food product. It is preferable that the foodstuff according to the present invention is a proteinaceous foodstuff.

In a preferred embodiment, the foodstuff is a meat foodstuff, i.e. a foodstuff which comprises meat, such as the meat referred herein, such as meat selected from the group consisting of pork (pig), beef, turkey, chicken, lamb or fish. Preferably meat foodstuffs are pork and chicken, most preferably pork.

In one embodiment, the meat foodstuff is the meat substrate referred to herein, which may be prepared by the comminuting methods referred to herein.

The term “food product” as used herein refers to an edible composition which consists or comprises of one or more “foodstuffs”. Typically, a food product comprises several “foodstuffs” which are combined and processed into a food product which are purchased (or eaten) directly by the consumer. In this regards, in one aspect a “food product” refers to a processed foodstuff.

In one embodiment, the protein-cross-linked proteinaceous product referred to herein is a (meat) food product, or is subsequently used in the preparation of a meat food product.

In a non limiting example, the food product may be selected from one or more of the following food product categories:
Cooked meat—For example ham, loin, picnic shoulder, bacon, pork belly.
Dry & semi-dry cured meat—Fermented products, dry-cured and fermented with starter cultures, for example dry sausages, salami, pepperoni, dry ham
Emulsified products for cold and hot consumption, for example mortadella, frankfurter, luncheon meat, pâté
Fish & seafood—shrimps, salmon, reformed fish products, frozen cold-packed fish.
Fresh meat muscle—Whole injected meat muscles, for example loin, shoulder ham, marinated meat.
Ground/restructered fresh meat—Reformed meat. Upgraded cut-away meat by cold-setting gel or binding, for example raw, uncooked loin chops, steaks, roasts, fresh sausages, beef burgers, meat balls, pelmeni.
Poultry products—For example chicken or turkey breast or reformed poultry, chicken nuggets, chicken sausages.
Retorted products—Autoclaved meat products, for example picnic ham, luncheon meat, emulsified products.
Vegetarian & meat analogues—For example vegetarian sausages, nuggets, burgers

The present invention utilises a first enzyme to remove inhibitors of a second enzyme from a proteinaceous substrate (composition). The first enzyme treatment may be performed prior to or even during the second enzyme treatment. The invention therefore relates to an enzyme system, wherein the enzyme system comprises a first enzyme and a second enzyme, wherein the first enzyme is capable of removing the inhibitor of a second enzyme from a substrate, such as a proteinaceous substrate.

In a preferred embodiment, the invention provides for an enzyme system comprising a first enzyme and a second enzyme, in which the first enzyme is an enzyme capable of removing free thiols from a proteinaceous substrate, and the second enzyme is a tyrosinase.

In one embodiment, the first enzyme and or the second enzyme is in isolated form.

The term “isolated” refers to the isolation of the enzyme from the environment it is naturally found in.

In one embodiment, the first enzyme and or the second enzyme is in purified form.

It is preferred that purified first and/or enzymes are used, i.e. the enzymes are purified prior to being added to the composition of the invention. Enzyme purity is preferably determined using SDS-PAGE and densitometry. A purified enzyme is at least 10% pure, such as at least 20% pure, such as at least 30% pure, such as at least 40% pure, such as at least 50% pure. It is recognized that a purified enzyme may however be formulated with other proteins, for example mixed with protein stabilizers such as BSA or other enzymes, the assessment of enzyme purity therefore excludes proteins added to the enzyme after purification. For the purpose of the enzyme system where both the first and second enzymes are present within the same compartment (1 pot system), the purity of the each (first or second) enzyme is calculated as if the other enzyme were not present.

In one embodiment, the first and second enzyme are present within the same enzyme composition.

However, in one embodiment, the first enzyme is isolated from the second enzyme (prior to use).

In this regards the enzyme system may be a single pot (part) system or a multiple pot (parts) (e.g. two) pot system.

The enzyme system may therefore be provided in the form of a kit, said kit comprising a first pot (part) which comprises the first enzyme, and a second pot (part) which comprises the second enzyme.

The term pot or part, as used above, refers to a single compartment, isolated from the other pot/parts/or compartment(s).

The amount of the first enzyme present in the enzyme system (or individual pot) may, for example be at least 0.001 mg/g, such as at least 0.01 mg/g, such as at least 0.1 mg/g, such as at least 1 mg/g, such as at least 3 mg/g, such as at least 5 mg/g, such as at least 10 mg/g. Suitably a dosage of about 15 mg/g or higher may be appropriate, for example up to 25 mg/g, 50 mg/g, up to 100 mg/g, up to 200 mg/g or even up to 300 mg/g.

The amount of the second enzyme present in the enzyme system (or individual pot) may, for example be at least 0.001 mg/g, such as at least 0.01 mg/g, such as at least 0.1 mg/g, such as at least 1 mg/g, such as at least 3 mg/g, such as at least 5 mg/g, such as at least 10 mg/g, such as at least 50 mg/g, such as at least 100 mg/g, such as at least 200 mg/g, or even higher, such as up to 500 mg/g, such as up to 400 mg/g, or, for example up to 25 mg/g, 50 mg/g, up to 100 mg/g, up to 200 mg/g or even up to 300 mg/g.

In one embodiment, the enzyme system is in the form of a food processing aid, for the enzymatic treatment of proteinaceous food products.

The enzyme system or systems may any other ingredient typically used in enzyme formulation, such as food enzyme composition ingredients or carriers—for example it may contain an extender such as maltodextrin, a carbohydrate based material, a silica based material, a protein, a protein hydrolysate and/or an other protein based material, such as BSA.

In one embodiment, by way of example, 10 mg of the mix could contain 0.015 mg SOX, 2 mg tyrosinase and 7.985 mg extender. Alternatively 100 mg of the mix could contain 0.015 mg SOX, 2 mg tyrosinase and 97.985 mg extender. In the first case one would add 10 mg of the mix pr. gram of protein to be treated, in the latter case one would add 100 mg of the mix pr. g of protein to be treated. If one assumes that the protein content of the meat product to be treated is 3%, one would in the first case add 300 mg of enzyme mix to 1 kg of meat product in the latter case one would add 3000 mg pr kg of meat product.

Applicants note that using protein as an extender could further have the benefit of adding additional substrate for the tyrosinase leading to further cross-linking.

In one embodiment the invention provides for the use of the enzyme system of the invention for the preparation of a proteinaceous foodstuff which has enhanced water retention; and/or

In one embodiment the invention provides for the use of the enzyme system of the invention for the preparation of a proteinaceous foodstuff which has enhanced gel-strength; and/or

In one embodiment the invention provides for the use of the enzyme system of the invention for the preparation of a proteinaceous foodstuff which has enhanced texture or mouth-feel.

The product of the invention, such as the protein cross-linked proteinaceous product, may have an improved gel strength as compared to either the proteinaceous substrate, or a comparative product prepared without a treatment step a). In one embodiment, the improvement of gel strength is at least 1 g such as at least 2 g, at least 3 g, at least 4 g, at least 5 g.

The product of the invention, such as the protein cross-linked proteinaceous product, may have an improved water holding capacity as compared to either the proteinaceous substrate, or a comparative product prepared without a treatment step a). In one the water holding capacity has been improved by at least 1%, such as at least 5%, such as at least 10%, such as at least 20%.

The degree of protein cross-linking of the product of the invention, such as the protein cross-linked proteinaceous product, may have a degree of protein cross-linking of at least 5%, such as at least 10%, such as at least 20%, such as at least 30%, such as at least 40%, such as at least 50%, such as at least 60%, such as at least 70%, such as at least 80%, such as at least 90%, as compared to a non-treated proteinaceous substrate. The degree of protein cross-linking may be obtained by densitometry analysis of SDS-PAGE (reducing conditions may be used) of the protein extracts obtained from the product of the invention, as compared to the proteinaceous substrate.

In one embodiment, the degree of protein cross linking of the myosin protein present in the product may have a degree of protein cross-linking of at least 5%, such as at least 10%, such as at least 20%, such as at least 30% such as at least 40%, such as at least 50%, such as at least 60%, such as at least 70%, such as at least 80%, such as at least 90%, as compared to a non-treated proteinaceous substrate.

Enzyme preparations are produced in varying degrees of purity from animal, plant, and microbial sources. They may consist of whole killed cells, parts of cells, or cell-free extracts. They may also contain carriers, solvents, preservatives, and antioxidants. The enzyme preparations may be formulated as liquid, semi-liquid, or dry solid preparations. Food enzyme preparations have traditionally been added directly to food during processing. For many applications, the components of the preparation remain in the processed food product. In recent years, enzymes immobilized on solid supports have gained importance. Immobilized enzyme preparations may range from those that contain a highly specific, purified enzyme, to those that contain whole killed cells or structurally intact viable cells. For some enzymatic processes, co-immobilization of enzymes and cells may be advantageous. Immobilized enzyme preparations are not intended to become food components.

It is therefore recognised that in one aspect the first enzyme and or the second enzyme are immobilised. However, in considering the activity of the second enzyme it is preferably that the second enzyme is not immobilised, i.e. the enzyme, possibly in inactive (e.g. heat deactivated form), remains in the food product.

### Further Embodiments and Aspects

Embodiment 1. An enzyme system comprising a first enzyme and a second enzyme, in which the first enzyme is an enzyme capable of removing free thiols from a proteinaceous substrate, and the second enzyme is a tyrosinase.

Embodiment 2. The enzyme system according to embodiment 1, in which the first enzyme and or the second enzyme is in isolated or purified form.

Embodiment 3. The enzyme system according to any of the preceding embodiments, in which the first enzyme is an oxidase.

Embodiment 4. The enzyme system according to embodiment 1 in which the first enzyme has an EC code 1.8.3.2. or 1.8.3.3.

Embodiment 5. The enzyme system according to embodiment 1 in which the first enzyme is sulfhydryl oxidase.

Embodiment 6. The enzyme system according to embodiment 1 in which the first enzyme is laccase

Embodiment 7. The enzyme system according to embodiment 1 in which the first enzyme is glutathione oxidase

Embodiment 8. The enzyme system according to embodiment 1 in which the first enzyme is disulfide isomerase.

Embodiment 9. The enzyme system according to embodiments 1 to 8, in which the first and second enzyme are present within the same enzyme composition.

Embodiment 10. The enzyme system according to any of the preceding embodiments, in which the first enzyme is isolated from the second enzyme prior to use.

Embodiment 11. The enzyme system according to embodiment 10, wherein the enzyme system is provided in the form of a kit, said kit comprising a first pot which comprises the first enzyme, and a second pot which comprises the second enzyme.

Embodiment 12. The enzyme system according to any one of the preceding embodiments, wherein the first enzyme is present in the enzyme system, prior to use, at a concentration of between 0.001 and 300 mg/g.

Embodiment 13. The enzyme system according to any one of the preceding embodiments, wherein the second enzyme is present in the enzyme system, prior to use, at a concentration of between 0.001 and 500 mg/g.

Embodiment 14. The enzyme system according to any one of the preceding embodiments, wherein the enzyme system is in the form of a food processing aid, for the enzymatic treatment of proteinaceous food products.

Embodiment 15. The enzyme system according to embodiment 14, wherein the enzyme system comprises a further component, such as one or more food enzyme composition ingredients and/or carriers.

Embodiment 16. The use of the first enzyme according to any one of the preceding embodiments, for reducing the concentration of free thiols in a proteinaceous substrate.

Embodiment 17. The use of the first enzyme according to embodiment 16, in which the proteinaceous substrate is a proteinaceous foodstuff, which preferably comprises myosin.

Embodiment 18. The use of the first enzyme according to embodiment 17, in which the proteinaceous food substrate contains or is derived from animal meat, such as mammalian, bird, or fish.

Embodiment 19. The use of the first enzyme according to embodiment 17, in which the proteinaceous food substrate contains or is derived from meat selected from the group consisting of pork, lamb, chicken, beef, turkey, cod, kangaroo, ostrich, shark.

Embodiment 20. The use of the first enzyme according to embodiment 17, in which the proteinaceous food substrate is pork, lamb, and chicken, most preferably pork.

Embodiment 21. The use of the first enzyme according to any of embodiments 16 to 20, which results in a reduction in the free thiol concentration of the proteinaceous substrate is of at least 5%.

Embodiment 22. The use of the first enzyme according to any of embodiments 16 to 21, which results in a reduction in the free thiol concentration of the proteinaceous substrate by at least 1 μM.

Embodiment 23. The use of the first enzyme according to any of embodiments 16 to 22, in which the reduction in free thiol concentration of the proteinaceous substrate is sufficient to enhance the ability of a second enzyme to cross link the protein present in the substrate, such as myosin.

Embodiment 24. The use of the first enzyme according to embodiment 23, in which the second enzyme is as defined in any one of embodiments 1-15.

Embodiment 25. A method for the preparation of a protein cross-linked foodstuff, said method comprising the steps:
a. treatment of a proteinaceous foodstuff with a first enzyme capable of degrading or oxidising free thiols;
b. treatment of the proteinaceous foodstuff with a second enzyme;

wherein step a) is performed prior to, or simultaneous to step b), and wherein step b) results in the cross-linking of proteins present in said proteinaceous food product.

Embodiment 26. A method according to embodiment 25 in which the enzyme capable of degrading or oxidising free thiols is as according to the first enzyme as defined in any one of embodiments 1-15.

Embodiment 27. A method according to embodiment 25 or 26, wherein step a) comprises the use of the first enzyme as according to any one of embodiments 16-24.

Embodiment 28. A method according to any one of embodiments 25-27, wherein the step a) results in a reduction in the free thiol concentration of the proteinaceous food substrate of at least 5%.

Embodiment 29. A method according to any one of embodiments 25-28, wherein the step a) results in a reduction in the free thiol concentration of the proteinaceous food substrate by at least 1 μM.

Embodiment 30. A method according to any one of embodiments 25-29, wherein the step a) results in a reduction in free thiol concentration of the proteinaceous food substrate which is sufficient to enhance the ability of the second enzyme to cross link the protein present in the food substrate, such as myosin.

Embodiment 31. A method according to any one of embodiments 25-30, wherein the second enzyme is as defined in any one of embodiments 1-15.

Embodiment 32. A method according to any one of embodiments 25-31, wherein the proteinaceous food product is as defined in any one of embodiments 17-20.

Embodiment 33. A method according to any one of embodiments 25-32, wherein the proteinaceous foodstuff comprises myosin, and the degree of cross-linking of the myosin protein present in the treated proteinaceous food products, subsequent to steps a) and b) is at least 5%.

Embodiment 34. A method according to any one of embodiments 25-33, wherein the gel strength or water holding capacity, or both, of the proteinaceous foodstuff after steps a) and b) are enhanced compared to the untreated proteinaceous food product.

Embodiment 35. The foodstuff prepared by any one of embodiments 25-34.

Embodiment 36. The proteinaceous foodstuff according to embodiment 35, wherein the concentration of free thiols present in said food product is no greater than 15 μM, or no greater than 10 μM, or no greater than 5 μM.

Embodiment 37. The proteinaceous according to embodiment 35 to 36 in which the gel strength has been enhanced by at least 2 g.

Embodiment 38. The proteinaceous foodstuff according to any of embodiments 35 to 37 in which the water holding capacity has been enhanced by at least 5%, such as at least 10%.

Embodiment 39. The proteinaceous foodstuff according to any of embodiments 35 to 38 in which at least 10% of the total myosin is cross linked.

Embodiment 40. The proteinaceous foodstuff according to any of embodiments 35 to 39 in which the concentration of free thiols has been reduced by at least 2 μM.

Embodiment 41. The proteinaceous foodstuff according to any of embodiments 35 to 40 in which the proteinaceous foodstuff is derived from animal meat, such as mammalian, bird, or fish.

Embodiment 42. The proteinaceous foodstuff according to any of embodiments 35 to 41 in which the food product is free from artificial additives, particularly additives such as phosphates.

Embodiment 43. A processed food product comprising the proteinaceous foodstuff according to any one of embodiments 35 to 42.

Embodiment 44. The processed food product according to embodiment 43, wherein the food product is selected from the group consisting of cooked meat, dry & semi-dry cured meat products, fermented products, emulsified products, fish & seafood products, fresh meat muscle, ground/restructered fresh meat, reformed meat, poultry products, retorted products, autoclaved meat products, vegetarian & meat analogue products.

Embodiment 45. The use of the enzyme system according to any one of embodiments 1-15 for the preparation of a proteinaceous foodstuff which has enhanced water retention.

Embodiment 46. The use of the enzyme system according to any one of embodiments 1-15 for the preparation of a proteinaceous foodstuff which has enhanced gel-strength.

Embodiment 47. The use of the enzyme system according to any one of embodiments 1-15 for the preparation of a proteinaceous foodstuff which has enhanced texture or mouth-feel.

Aspect 1. A method for the preparation of a cross-linked proteinaceous substrate, said method comprising the steps:
a. treatment of a proteinaceous substrate comprising free thiols with a first enzyme capable of removing free thiols;
b. treatment of the proteinaceous substrate with a second enzyme, the activity of which is inhibited by free thiols;

wherein step a) is performed prior to, or simultaneous to step b), and wherein step b) results in the cross-linking of proteins present in said proteinaceous substrate and wherein the first enzyme and the second enzyme is not the same enzyme.

Aspect 2. The method according to aspect 1, wherein the second enzyme is selected from the group consisting of a tyrosinase, laccase, lipoxygenase and protein lysin 6-oxidase (lysyl oxidase).

Aspect 3. The method according to aspect 2, wherein the second enzyme is selected from the group consisting of lipoxygenase and protein lysin 6-oxidase (lysyl oxidase).

Aspect 4. The method according to any one of aspects 1-3, wherein the second enzyme is laccase.

Aspect 5. The method according to any one of aspects 1-3, wherein the second enzyme is a tyrosinase.

Aspect 6. The method according to any one of aspects 1-5, in which the first enzyme and/or the second enzyme is in isolated or purified form.

Aspect 7. The method according to any one of aspects 1-6, in which the first enzyme is an oxidase.

Aspect 8. The method according to any one of aspects 1-6, wherein the first enzyme is not laccase.

Aspect 9. The method according to any one of aspects 1-6, in which the first enzyme has an EC code 1.8.3.2. or 1.8.3.3.

Aspect 10. The method according to any one of aspects 1-6, in which the first enzyme is sulfhydryl oxidase.

Aspect 11. The method according to any one of aspects 1-6, in which the first enzyme is glutathione oxidase

Aspect 12. The method according to any one of aspects 1-6, in which the first enzyme is disulfide isomerase.

Aspect 13. The method according to any one of aspects 1-12, in which the proteinaceous substrate is a proteinaceous foodstuff, which preferably comprises myosin.

Aspect 14. The method according to aspect 13, in which the proteinaceous foodstuff contains or is derived from animal meat, such as mammalian, bird, or fish.

Aspect 15. The method according to aspect 14, in which the proteinaceous foodstuff contains or is derived from meat selected from the group consisting of pork, lamb, chicken, beef, turkey, cod, kangaroo, ostrich, shark.

Aspect 16. The method according to aspect 15, in which the proteinaceous foodstuff is selected from the group consisting of pork, lamb, and chicken, most preferably pork.

Aspect 17. The method according to any one of aspects 1-16, in which the first and second enzyme are present within the same enzyme composition.

Aspect 18. The method according to any one of aspects 1-17, in which the first enzyme is isolated from the second enzyme prior to use.

Aspect 19. The method according to any one of aspects 1-18, wherein the first enzyme and the second enzyme is provided as an enzyme system in the form of a kit, said kit comprising a first pot which comprises the first enzyme, and a second pot which comprises the second enzyme.

Aspect 20. The method according to aspect 19, wherein the first enzyme is present in the form of an enzyme system, prior to use, at a concentration of between 0.001 and 300 mg/g.

Aspect 21. The method according to any one of aspects 19-20, wherein the second enzyme is present in the form of an enzyme system, prior to use, at a concentration of between 0.001 and 500 mg/g.

Aspect 22. The method according to any one of aspects 19-21, wherein the enzyme system is in the form of a food processing aid, for the enzymatic treatment of proteinaceous food products.

Aspect 23. The method according to aspect 22, wherein the enzyme system comprises a further component, such as one or more food enzyme composition ingredients and/or carriers.

Aspect 24. The method according to any one of aspects 1-23, wherein the removal of free thiols in step a) results in a reduction in the free thiol concentration of the proteinaceous substrate of at least 5%.

Aspect 25. A method according to any one of aspects 1-24, wherein the removal of free thiols in step a) results in a reduction in the free thiol concentration of the proteinaceous substrate by at least 1 μM.

Aspect 26. Use of the first enzyme as defined in any one of the preceding aspects, for reducing the concentration of free thiols in a proteinaceous substrate.

Aspect 27. The use of the first enzyme according to aspect 26, in which the proteinaceous substrate is a proteinaceous foodstuff, which preferably comprises myosin.

Aspect 28. The use of the first enzyme according to aspect 27, in which the proteinaceous foodstuff contains or is derived from animal meat, such as mammalian, bird, or fish.

Aspect 29. The use of the first enzyme according to aspect 28, in which the proteinaceous foodstuff contains or is derived from meat selected from the group consisting of pork, lamb, chicken, beef, turkey, cod, kangaroo, ostrich, and shark.

Aspect 30. The use of the first enzyme according to aspect 29, in which the proteinaceous foodstuff is selected from the group consisting of pork, lamb, and chicken, most preferably pork.

Aspect 31. The use of the first enzyme according to any of aspects 26 to 30, which results in a reduction in the free thiol concentration of the proteinaceous substrate of at least 5%.

Aspect 32. The use of the first enzyme according to any of aspects 26 to 31, which results in a reduction in the free thiol concentration of the proteinaceous substrate by at least 1 μM.

Aspect 33. The use of the first enzyme according to any of aspects 26 to 32, in which the reduction in free thiol concentration of the proteinaceous substrate is sufficient to enhance the ability of a second enzyme to cross link the protein present in the substrate, such as myosin.

Aspect 34. The use of the first enzyme according to aspect 33, in which the second enzyme is as defined in any one of aspects 1-25.

Aspect 35. A method for improving the enzymatic activity on a proteinaceous substrate comprising free thiols by a second enzyme, the activity of which is inhibited by the presence of free thiols, said method comprising treatment of the proteinaceous substrate with a first enzyme capable of removing free thiols; wherein the treatment with the first enzyme is performed prior to, or simultaneous with a treatment with the second enzyme and wherein the first enzyme and the second enzyme is not the same enzyme.

Aspect 36. The method according to aspect 35, wherein the enzymatic activity results in the cross-linking of proteins present in said proteinaceous substrate.

Aspect 37. The method according to any one of the aspects 35-36, wherein the first enzyme is as defined in any one of aspects 1-26.

Aspect 38. The method according to any one of aspects 35-37, wherein the second enzyme is selected from the group consisting of tyrosinase, laccase, lipoxygenase, galactose oxidase, protein lysin 6-oxidase (lysyl oxidase), galactolipase and lysophospholipase,

Aspect 39. The method according to any one of aspects 35-38, wherein the proteinaceous substrate is as defined in any one of aspects 1-26.

Aspect 40. A method for the preparation of a protein cross-linked foodstuff, said method comprising the steps:
a. treatment of a proteinaceous foodstuff with a first enzyme capable of degrading or oxidising free thiols;
b. treatment of the proteinaceous foodstuff with a second enzyme;

wherein step a) is performed prior to, or simultaneous to step b), and wherein step b) results in the cross-linking of proteins present in said proteinaceous food product.

Aspect 41. The method according to aspect 40 in which the enzyme capable of degrading or oxidising free thiols is as defined in any one of aspects 1-25.

Aspect 42. The method according to aspect 40 or 41, wherein step a) comprises the use of the first enzyme according to any one of aspects 26-34.

Aspect 43. The method according to any one of aspects 40-42, wherein the step a) results in a reduction in the free thiol concentration of the proteinaceous foodstuff of at least 5%.

Aspect 44. The method according to any one of aspects 40-43, wherein the step a) results in a reduction in the free thiol concentration of the proteinaceous foodstuff by at least 1 μM.

Aspect 45. The method according to any one of aspects 40-44, wherein the step a) results in a reduction in free thiol concentration of the proteinaceous foodstuff which is sufficient to enhance the ability of the second enzyme to cross link the protein present in the food substrate, such as myosin.

Aspect 46. The method according to any one of aspects 40-45, wherein the second enzyme is as defined in any one of aspects 1-25.

Aspect 47. The method according to any one of aspects 40-46, wherein the proteinaceous food product is as defined in any one of aspects 27-30.

Aspect 48. The method according to any one of aspects 40-47, wherein the proteinaceous foodstuff comprises myosin, and the degree of cross-linking of the myosin protein present in the treated proteinaceous food products, subsequent to steps a) and b) is at least 5%.

Aspect 49. The method according to any one of aspects 40-48, wherein the gel strength or water holding capacity, or both, of the proteinaceous foodstuff after steps a) and b) are enhanced compared to the untreated proteinaceous food product.

Aspect 50. The foodstuff prepared by any one of aspects 40-49.

Aspect 51. The proteinaceous foodstuff according to aspect 50, wherein the concentration of free thiols present in said food product is no greater than 15 μM, or no greater than 10 μM, or no greater than 5 μM.

Aspect 52. The proteinaceous according to aspect 50 to 51, in which the gel strength has been enhanced by at least 2 g.

Aspect 53. The proteinaceous foodstuff according to any of aspects 50 to 52, in which the water holding capacity has been enhanced by at least 5%, such as at least 10%.

Aspect 54. The proteinaceous foodstuff according to any of aspects 50 to 53, in which at least 10% of the total myosin is cross linked.

Aspect 55. The proteinaceous foodstuff according to any of aspects 50 to 54, in which the concentration of free thiols has been reduced by at least 2 μM.

Aspect 56. The proteinaceous foodstuff according to any of aspects 50 to 55, in which the proteinaceous foodstuff is derived from animal meat, such as mammalian, bird, or fish.

Aspect 57. The proteinaceous foodstuff according to any of aspects 50 to 56, in which the food product is free from artificial additives, particularly additives such as phosphates.

Aspect 58. A processed food product comprising the proteinaceous foodstuff according to any one of aspects 50 to 57.

Aspect 59. The processed food product according to aspect 58, wherein the food product is selected from the group consisting of cooked meat, dry & semi-dry cured meat products, fermented products, emulsified products, fish & seafood products, fresh meat muscle, ground/restructered fresh meat, reformed meat, poultry products, retorted products, autoclaved meat products, vegetarian & meat analogue products.

Aspect 60. The use of the method according to any one of aspects 1-25 for the preparation of a proteinaceous foodstuff which has enhanced water retention.

Aspect 61. The use of the method according to any one of aspects 1-25 for the preparation of a proteinaceous foodstuff which has enhanced gel-strength.

Aspect 62. The use of the method according to any one of aspects 1-25 for the preparation of a proteinaceous foodstuff which has enhanced texture or mouth-feel.

The invention will now be further described by way of the following non-limiting examples.

### EXAMPLE

Materials and methods

Ingredients:

Minced meat from pork (3-7% fat), beef (9-12% fat), lamb (8-10% fat), chicken (3-6% fat), turkey (3-7% fat), and cod (fat content unknown) were all purchased at local stores. Sodium tripolyphosphate (STPP) Tyrosinase (TrTyr2; VTT, Finland, 1067 nkat/ml according to supplier), TGase (Activa MP; Ajinomoto; 1666 nkat/g according to supplier), Sulfhydryl oxidase (Aspergillus spp/Aspergillus niger; 325 nkat/g; measured activity)

Protein Extractions:
1. Brine was made using 2% NaCl and 0.3% STPP
2. 20 g portions of meat were placed in 250 ml baffled shake flasks
3. 60 ml of brine were added to each shake flask.
4. The shake flasks were placed on a rotary shaker (Certomat

R, Braun Int.); 120 min at room temperature and 180 rpm
5. The meat-brine suspension was centrifuged for 60 min at 10000 g and 5° C.
6. The fat particles were removed by filtering the supernatant through a 0.8 mm grid
7. The meat-protein extract (supernatant) was stored at −18° C.

Gel Filtration/Dialysis

Dialysis was carried out in Slide-A-Lyzer Dialysis Cassette from Pierce (Order nr: 66810). The dialysis was carried out at 4° C. overnight with one shift of dialysis buffer.

Determination of Free Thiol Concentration

Ellmans Reagent I: 3 mM EDTA (0.558 g); 0.2M Tris (12,14 g) was dissolved in ca 400 ml water, and adjusted to pH to 8 with HCl and filled up to 500 ml with water. Ellmans Reagent II: DTNB (Sigma D-8130) 8 mG was dissolved in 20 ml Ellmans I and stored in the dark and used within 1 day.

For removal of protein bound thiol and for clarification of the sample: The meat extract (0.5 ml) and 0.5% Trichloraceticacid (0.5 ml) was mixed in an eppendorf tube. The tube was centrifuged 5 min at 10000 g. 0.3 ml supernatant was transferred to a new tube and neutralised with 40 μL 1M NaOH.

Analysis: 100 μL protein free sample was mixed with 170 μl Ellmans Reagent II in a microtiter tray well. The samples were mixed using a plate shaker. The plate was incubated at room temperature inside the microtiter plate reader (to keep in dark) and Abs₄₂₀ was measured exactly 2 min after addition of Ellmanns Reagent II.

Calculation of Thiol Concentration:

ε=Extinction coefficient

(2-nitro-5-thiobenzoate; NTB)=13600 M⁻¹*cm⁻¹

r=radius of well=0.35 cm

v=volume sample in well=0.27 mL

Light path=I=v/(ε*r²)=0.27/(ε*0.35²)=0.70 cm

Thiol concentration (mol/L)=Abs₄₂₀/I*ε)=Abs₄₂₀/(0.70*13600)

All determinations were made in duplicate.

Measurement of Sulfhydryl Oxidase Activity:

Measured as drop in dithiotreitol (DTT) concentration (according to above) after adding sulfhydryl oxidase. The activity is defined as nmol DTT oxidised per second.

Determination of Protein Content

Total nitrogen content in percent was determined according to the Kjeldahl method (Ma T S & Zuazaga G. Micro-Kjeldahl determination of nitrogen. Ind. Eng. Chem. (Analytical Edition) 14:280-2, 1942). Protein content was determined by multiplying the total nitrogen % by the factor 6.25 (http://www.foodcomp.dk/fvdb_aboutfooddata_proximates.asp#Protein).

Sample Preparation/Enzyme Treatment for SDS-PAGE Analysis

Enzyme treatment of the protein extracts prior to SDS-PAGE analysis was done as follows: Tyrosinase was added to a final concentration of 15.5 nkat/ml extract (corresponding to 0.015 mg tyrosinase per ml extract) and incubated for 60 min at 40° C. TGase was added to a final concentration of 2.5 nkat/ml extract (corresponding to 0.015 mg TGase pr. ml extract) and incubated for 60 min at 40° C. sulfhydryl oxidase was added to a final concentration of 0.0033 (low sulfhydryl oxidase) or 0.033 (high sulfhydryl oxidase) nkat/ml. It was always added prior to or simultaneously with other enzyme treatment. If added prior to other enzyme treatment, an incubation time of 60 min at room temperature was applied.

SDS-Page Analysis:

SDS-PAGE was carried out on Pre-cast Novex Bis-Tris gels (Invitrogen, Carlsbad Calif., USA) according to manufacturers' protocol. For non-reducing SDS-PAGE, DTT was left out of the sample buffer.

Tyrosinase Spot Assay and Test of Inhibition

Spot assay of tyrosinase activity was determined by adding 11 nkat of tyrosinase to 1 ml of skimmed milk. Red colour formation within 5-10 seconds is an indication of high of tyrosinase activity. To test for tyrosinase inhibition, either the inhibitor is mixed with tyrosinase before addition to the milk or the inhibitor is added to the milk prior to tyrosinase addition. The inhibitors tested were free cysteine at final concentrations of 1 or 0.1 mM or meat extracts diluted 10 times in the final sample.

Production of Meat Protein Gels for Texture and WHC Assessment

Heat induced meat protein gels were produced according to the following method:
1. 12 g meat-protein extract was placed in 50 ml screw top plastic centrifuge tubes
2. Enzyme treatment: Either 180 μL TrTyr2 (1067 nkat/ml)+180 μL water, 180 μL sulfhydryl oxidase (1 mg/ml)+180 μL TrTyr2 (1067 nkat/ml), 180 μL sulfhydryl oxidase (1 mg/ml)+180 μL water or 360 μL water (reference) was added to the meat-protein extract
3. The samples were incubated for 1 h at 40° C.
4. A gel was produced by heating at 80° C. for 60 min
5. The gel was stored at 5° C. overnight
6. The gel was tempered at 35° C. for 2 hours
7. A TPA analysis was carried out on the gel in the tube using Texture analyser (TI-XT2, Stable micro systems) with the following settings: Pre speed 2 mm/sek, Test sped 5 mm/sek, trigger force: 3 g, travel distance 3 mm, time between compressions 5 sec, Load cell 5 kg, probe: P0.5 (5 mm ebonite)
8. A cross was cut in the gel and the tubes were centrifuged (Hettich Rotina 46) for 10 min at 4000 rpm
9. Supernatant was poured off and weighed

The water holding capacity (WHC) was calculated as follows:

$$WHC = 1 - \frac{{W}_{R}}{{W}_{Tot}}$$

W_{R} is the weight of water released from the gel and W_{Tot} is the total weight of the gel

### Results

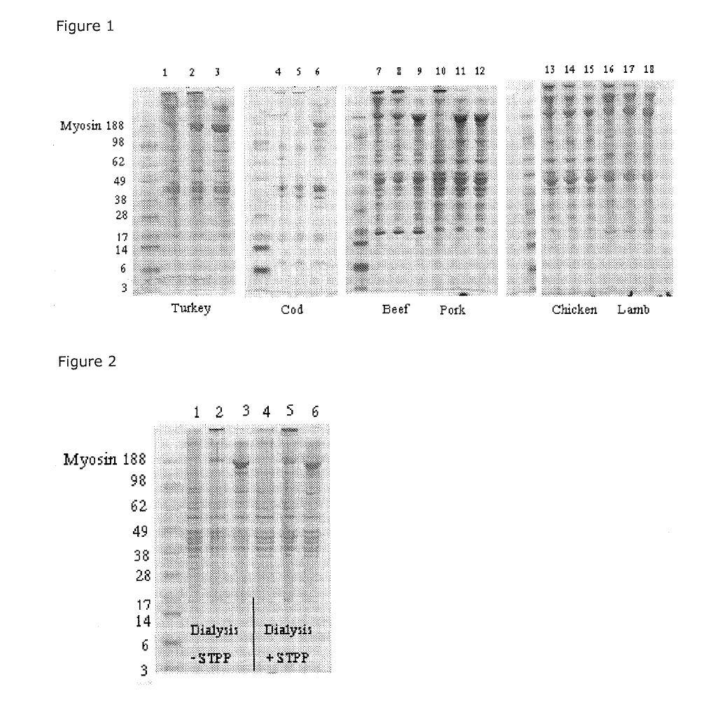

FIG. 1 shows SDS-PAGE gels of all the meat protein extracts, which were produced. Each extract was treated with TGase (first lane of a specific extract), Tyrosinase (second lane of a specific extract) and no enzyme (third lane of a specific extract). The band at a MW of around 188 KDa is myosin.

FIG. 2 shows SDS-PAGE analysis of extracts from pork, which were dialysed prior to electrophoresis. For the first three lanes, the extract was dialysed against a brine solution similar to the brine used for the extraction except that it contained no STPP. For lanes 4-6, the extract was dialysed against a brine solution identical to the one used for extraction. As in FIG. 1, extracts were treated with both TGase (lane 1 and 4) and TrTyr2 (lane 2 and 5).

Table 1 lists the protein and free thiol concentration measured in each of the extracts (average of two determinations). The results show large differences in both protein and free thiol content of the different extracts. There does not seem to be any correlation between protein content and free thiol concentration. Free thiol content of pork and beef extracts after treatment with 0.033 nkat/ml sulfhydryl oxidase for 60 minutes was also determined.

TABLE 1
Protein content and free thiol concentration
of the different protein extracts.

| | | Free thiol after | |
|---|---|---|---|
| | | sulfhydryl | |
| | Free thiol | oxidase- | Protein |
| | (μM) | treatment (μM) | % |
| Pork | 18.4 | 2.6 | 3.1 |
| Beef | 6.0 | 2.3 | 2.5 |
| Turkey | 6.4 | — | 2.1 |
| Chicken | 14.5 | — | 3.3 |
| Lamb | 12.3 | — | 2.6 |
| Cod | 3.4 | — | 1.9 |

Activity spot tests showed that free cysteine down to a concentration of about 10 μM gave a pronounced inhibition of the tyrosinase. The cysteine and enzyme were in contact with each other for about 1 min before adding the substrate.

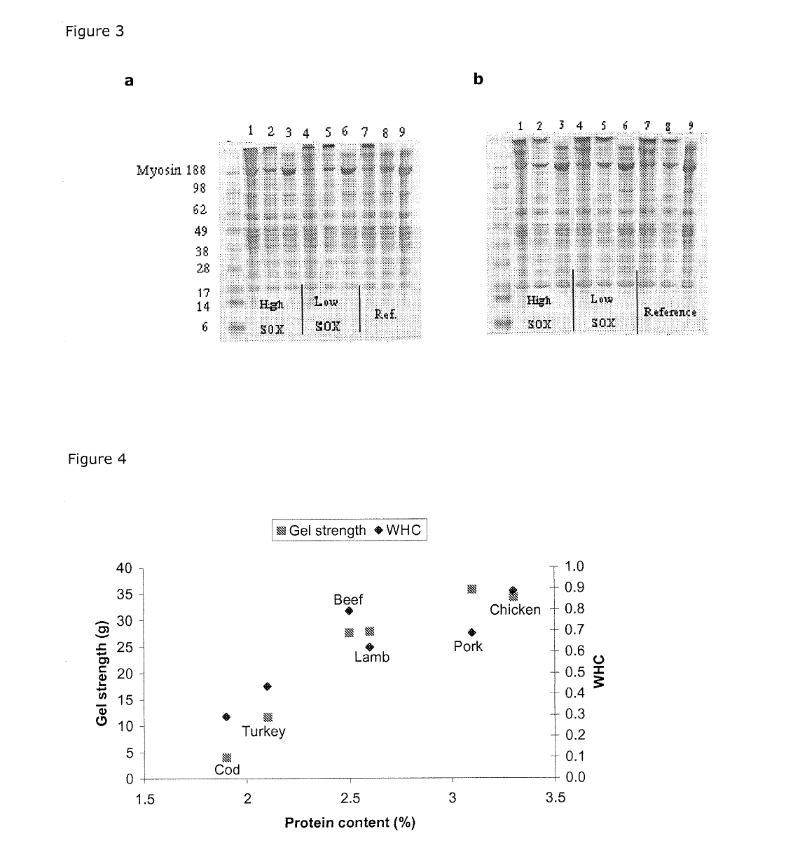

FIG. 3 shows SDS-PAGE analysis of pork (a) and beef (b) extracts, which were treated with sulfhydryl oxidase to remove free thiols before addition of cross-linking enzyme.

FIG. 4 illustrates that both gel-strength and WHC of gels made from the different meat extracts are positively correlated with protein content. Note that this figure only depicts reference samples (no enzyme treatment). The gel made from cod extract was inhomogeneous, which gives large variations in gel strength measurement. However, it was consistently lower than for the other gels.

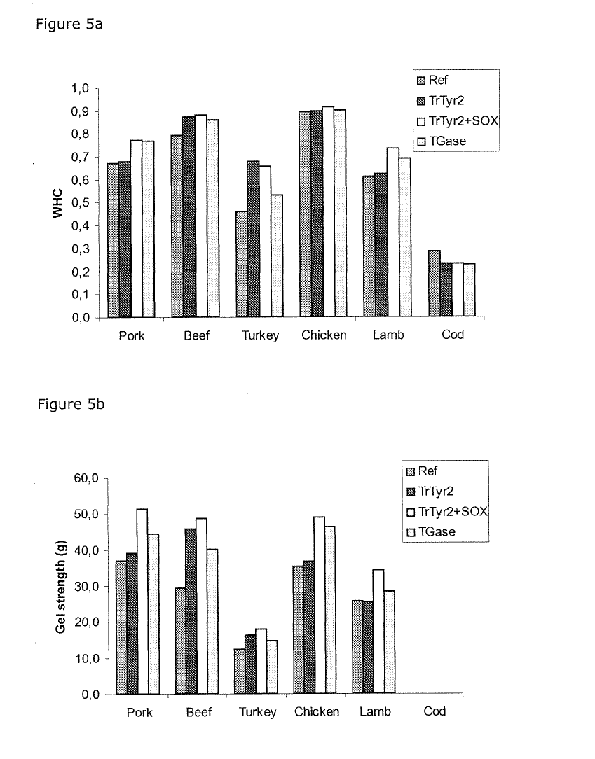

FIGS. 5a and b, respectively, depicts gel strength and WHC of heat induced gels from different protein extracts with various enzymatic treatments. Remarkably positive effects on gel strength were observed on gels from pork, chicken and lamb when treated with both sulfhydryl oxidase and TrTyr2. For gels made from beef and turkey extracts, treatment with sulfhydryl oxidase+ Tyrosinase gave similar results as treatment with tyrosinase alone. Gel strength results for cod were omitted due to very inhomogeneous gels. The tendencies observed for gel WHC were similar to gel strength except that WHC for chicken gels was unaltered regardless of treatment. WHC for cod could be measured even though the gels were inhomogeneous. TGase treatments were run for reference.

The experiments with pork extract and the combination of sulfhydryl oxidase and tyrosinase were repeated with similar results. However, the second experiment was run with a new protein extract, giving different levels of both gel strength and WHC. Hence, the results were not included in FIG. 5. Also, a sample was run with sulfhydryl oxidase alone. In this case no difference on gel strength and WHC was observed compared to the reference sample.

### Discussion

The effects of tyrosinase treatment on meat protein extracts from different species of animals are not similar (FIG. 1). Most noticeably, no cross-linking seems to take place upon tyrosinase treatment in extracts from pork (compare lane 11—tyrosinase treated, with lane 12—reference), while in extracts from e.g. beef, HMW protein bands in the top of the gel are observed upon tyrosinase treatment, indicating protein cross-linking (compare lane 8—tyrosinase treated, with lane 9—reference). Concomitantly there is a reduction in the intensity of the myosin band (at 188 KDa), which indicates that it is mainly this protein, which is cross-linked. Except for pork, tyrosinase treatment produces high MW protein aggregates in extracts from all species. However, the efficiency of the cross-linking activity seems to vary. In cod extract the myosin band disappears entirely, indicating a very efficient cross-linking. While for beef, turkey, chicken and lamb there are varying degrees of myosin-band fading, indicating varying degrees of cross-linking efficiency.

TGase, a well-known protein cross-linking enzyme, is always included as a positive reference (lane 1, 4, 7, 13, 16 in FIG. 1). It is seen that TGase catalysed cross-linking is efficient in all extracts. In the results described herein, TGase activity was not influenced by any further treatment.

Myosin is a very conserved protein in the animal kingdom, and therefore the myosin substrate is considered to be equivalent between the different meat samples.

Applicants have identified that the low tyrosinase activity in meat substrates such as pork or chicken, is due to the presence of tyrosinase inhibition in the different extracts. This notion was substantiated by two simple tests: 1) Ten times dilution of the pork extract prior to tyrosinase treatment rendered the enzyme active (observed as a high MW band and myosin fading on SDS-PAGE; results not shown). 2) Tyrosinase makes milk turn red due to formation of quinones. This is used as a simple tyrosinase activity spot test. If tyrosinase was mixed with pork extract before addition to the milk, no colour formation was observed. Both tests indicate an inhibitory effect of the extract.

In addition Applicants have attempted to enhance the texture of protein gels made from protein extracts of minced pork, but it was observed that very high tyrosinase concentrations were necessary in order to obtain texture enhancements. This result also supports that tyrosinase is being inhibited from some component in the pork extract.

A dialysis of the pork extract was carried out (FIG. 2). It is seen that dialysis against the exact same solution as was used for extraction of the proteins rendered the tyrosinase active (compare lanes 5 and 6). This indicates that the inhibition originates from a low MW compound.

Phosphates are potential inhibitors of tyrosinase since they may collate with copper atoms, which are essential for catalytic activity of tyrosinases. The phosphate STPP is present at a level of 0.3% in all extracts. However, it is unlikely that STPP is the cause of inhibition, since it is still present after dialysis. Moreover, it is present in extracts from all species, also the ones where tyrosinase catalysed cross-linking is efficient. Nevertheless, pork extract was dialysed against a solution free of STPP. Consistent with the above mentioned, no further efficiency of tyrosinase cross-linking was observed (compare lane 2 and 5; one should not draw conclusions upon small differences in band intensities since the SDS sample solutions were viscous, reducing pipetting accuracy). Addition of extra copper also did not seem to enhance tyrosinase activity (results not shown).

A literature study revealed two potential inhibitors, which are likely to be encountered in the meat extracts. One is triglycerides with a total number of double bonds exceeding 4, the other is free cysteine.

First, attempts were made by different procedures to remove the potentially inhibitory lipids from the meat before extraction. These included washing with solvents (hexane, acetem, tributyrin, or water) and treatment with lipase. In general, the results from these experiments were inconclusive, and by no means gave any indication that lipids were the main cause of the tyrosinase inhibition.

To follow up on the potential inhibitory effect of cysteine, tyrosinase activity was spotted with and without different concentrations of cysteine in the assay medium. Tyrosinase was clearly inhibited by cysteine at concentrations above 10 μM. Subsequently, the free thiol content (which includes free cysteine) in the different protein extracts was measured (Table 1). It was observed that the highest concentration of free thiol was present in the extract from pork and that the concentration (18.4 μM) was substantially above the limit of inhibition observed with cysteine. It is not known how much of the free thiol present in the samples originates from free cysteine. However, it is likely that the tyrosinase is inhibited by low MW free thiols in general, rather than free cysteine alone. In extracts from chicken and lamb, the free thiol concentration was also above the inhibitory level found for cysteine. These samples from chicken and Iamb extracts are also the ones where the least extensive fading of the myosin band occurs (FIG. 1), i.e. tyrosinase seems to be inhibited to some extent. Cod extract has the lowest content of free thiol and is also the extract in which the fading of the myosin band is most pronounced. All in all, this is strong evidence that free thiols are responsible for tyrosinase inhibition.

In an attempt to circumvent the inhibitory effect of free thiols, the extracts were treated with sulfhydryl oxidase, which oxidises free thiols to disulfide compounds. After treatment of extract from pork with a sulfhydryl oxidase (0.33 or 0.033 nkat/ml), tyrosinase was no longer inhibited, as seen from the formation of high MW protein aggregates and substantial fading of the myosin band on the SDS-PAGE analysis in FIG. 3a (compare lane 2, 5, and 8). Sulfhydryl oxidase alone did not cause any observable protein cross-linking (compare lane 3, 6 and 9). Applicants considered the possibility that the sulfhydryl oxidase produces disulfide cross-links between proteins, which may not observable on the gels in FIG. 3, due to the presence of the reducing agent DTT in the SDS sample buffer. Hence, a SDS-PAGE analysis was carried out under non-reducing conditions (without DTT). However, no cross-linking effect of sulfhydryl oxidase alone was observed (results not shown). For beef extract (FIG. 3b), tyrosinase was able to cross-link proteins, regardless of whether the extract had received sulfhydryl oxidase-treatment or not. This shows that sulfhydryl oxidase itself does not have a negative effect on tyrosinase activity.

Cross-linking of meat proteins may enhance textural and sensorial properties of meat products. In the earlier study it was found contrary to expectations that tyrosinase alone did not enhance gel strength and WHC of heat induced protein gels, made from pork extracts, notably. As a continuation of the above results, it was tested whether removal of free thiol in protein extracts could boost the functional effects expected by tyrosinase catalysed cross-linking. It was seen (FIG. 5) that tyrosinase alone may improve both strength and WHC of heat induced protein gels made from extracts with a low free thiol content (beef and turkey). Such improvements in gels produced from extracts with high thiol content (pork and chicken) were only possible if free thiol was removed (by sulfhydryl oxidase) before tyrosinase action.

There are very pronounced differences in the level of gel strength and WHC between the different extracts. This is a consequence of different protein contents in the extracts (FIG. 4)

### Conclusion

Protein extracts from different types of meat show very different characteristics with regard to tyrosinase catalysed cross-linking. The differences correlate to different levels of free thiol in the samples. Treating with sulfhydryl oxidase prior to tyrosinase treatment eliminated low efficiency of tyrosinase catalysed cross-linking in meat extracts with significant inhibition, such as extracts from pork.

Tyrosinase alone may improve both strength and WHC of heat induced protein gels made from extracts with a low free thiol content (beef and turkey). Such improvements in gels produced from extracts with high thiol content (pork and chicken) were only possible if free thiol was removed (by sulfhydryl oxidase) before tyrosinase action.

Applicants note that most meats have a detectable and in many cases significant free thiol content, therefore even in meats where it is possible to obtain a satisfactory gel strength or water holding capacity, the use of the sulfydryl oxidase enzyme will allow for a reduced dosage of tyrosinase, and/or shorter or more desirable enzymatic incubation conditions.

The concept is also applicable in whole meat products. This was shown by adding different combinations of SOX and tyrosinase to a model chicken sausage recipe containing minced chicken meat. It was shown that when tyrosinase was added subsequent to a SOX treatment, both product hardness and water holding capacity of the product increased significantly compared to the control product. This was not the case when any of the two enzymes were used alone. The tyrosinase-SOX combination produced significantly higher product hardness and water holding capacity than transglutaminase at a dose recommended by the supplier (results not shown).

1. A method for the preparation of a cross-linked proteinaceous substrate, said method comprising the steps:
a) treatment of a proteinaceous substrate comprising free thiols with a first enzyme capable of removing free thiols;
b) treatment of the proteinaceous substrate with a second enzyme, the activity of which is inhibited by free thiols;
wherein step a) is performed prior to, or simultaneous to step b), and
wherein step b) results in the cross-linking of proteins present in said proteinaceous substrate and
wherein the first enzyme and the second enzyme is not the same enzyme.

2. The method according to claim 1,
wherein the second enzyme is selected from the group consisting of a tyrosinase, laccase, lipoxygenase and protein lysin 6-oxidase (lysyl oxidase) or
wherein the second enzyme is laccase or
wherein the second enzyme is a tyrosinase or
in which the first enzyme and/or the second enzyme is in isolated or purified form or
wherein the first enzyme is an oxidase or
wherein the first enzyme is not laccase or
in which the first enzyme has an EC code 1.8.3.2. or 1.8.3.3 or
wherein the first enzyme is sulfhydryl oxidase or
wherein the first enzyme is glutathione oxidase or
wherein the first enzyme is disulfide isomerase.

3-12. (canceled)

13. The method according claim 1, in which the proteinaceous substrate is a proteinaceous foodstuff, which preferably comprises myosin.

14. The method according to claim 13, in which the proteinaceous foodstuff contains or is derived from animal meat, such as mammalian, bird, or fish.

15. The method according to claim 14, in which the proteinaceous foodstuff contains or is derived from meat selected from the group consisting of pork, lamb, chicken, beef, turkey, cod, kangaroo, ostrich, shark.

16. The method according to claim 15, in which the proteinaceous foodstuff is selected from the group consisting of pork, lamb, and chicken, most preferably pork.

17. The method according to claim 1,
in which the first and second enzyme are present within the same enzyme composition or
in which the first enzyme is isolated from the second enzyme prior to use.

18. (canceled)

19. The method according to claim 1, wherein the first enzyme and the second enzyme is provided as an enzyme system in the form of a kit, said kit comprising a first pot which comprises the first enzyme, and a second pot which comprises the second enzyme.

20. The method according to claim 19,
wherein the first enzyme is present in the form of an enzyme system, prior to use, at a concentration of between 0.001 and 300 mg/g or
wherein the second enzyme is present in the form of an enzyme system, prior to use, at a concentration of between 0.001 and 500 mg/g.

21. (canceled)

22. The method according to claim 19, wherein the enzyme system is in the form of a food processing aid, for the enzymatic treatment of proteinaceous food products.

23. The method according to claim 22, wherein the enzyme system comprises a further component, such as one or more food enzyme composition ingredients and/or carriers.

24. The method according to claim 1,
wherein the removal of free thiols in step a) results in a reduction in the free thiol concentration of the proteinaceous substrate of at least 5% or
wherein the removal of free thiols in step a) results in a reduction in the free thiol concentration of the proteinaceous substrate by at least 1 μM.

25-34. (canceled)

35. A method for improving the enzymatic activity on a proteinaceous substrate comprising free thiols by a second enzyme, the activity of which is inhibited by the presence of free thiols, said method comprising treatment of the proteinaceous substrate with a first enzyme capable of removing free thiols; wherein the treatment with the first enzyme is performed prior to, or simultaneous with a treatment with the second enzyme and wherein the first enzyme and the second enzyme is not the same enzyme.

36. The method according to claim 35, wherein the enzymatic activity results in the cross-linking of proteins present in said proteinaceous substrate.

37. The method according to claim 35,
wherein the first enzyme is as defined in claim 2 or
wherein the second enzyme is selected from the group consisting of tyrosinase, laccase, lipoxygenase, galactose oxidase, protein lysin 6-oxidase (lysyl oxidase), galactolipase and lysophospholipase or
wherein the proteinaceous substrate is as defined in claim 13.

38-39. (canceled)

40. A method for the preparation of a protein cross-linked foodstuff, said method comprising the steps:
a) treatment of a proteinaceous foodstuff with a first enzyme capable of degrading or oxidising free thiols;
b) treatment of the proteinaceous foodstuff with a second enzyme;
wherein step a) is performed prior to, or simultaneous to step b), and wherein step b) results in the cross-linking of proteins present in said proteinaceous food product.

41. The method according to claim 40
in which the enzyme capable of degrading or oxidising free thiols is as defined in claim 2 or
wherein the step a) results in a reduction in the free thiol concentration of the proteinaceous foodstuff of at least 5% or
wherein the step a) results in a reduction in the free thiol concentration of the proteinaceous foodstuff by at least 1 μM or
wherein the step a) results in a reduction in free thiol concentration of the proteinaceous foodstuff which is sufficient to enhance the ability of the second enzyme to cross link the protein present in the food substrate, such as myosin or
wherein the second enzyme is as defined in claim 2 or
wherein the proteinaceous foodstuff comprises myosin, and the degree of cross-linking of the myosin protein present in the treated proteinaceous food products, subsequent to steps a) and b) is at least 5% or
wherein the gel strength or water holding capacity, or both, of the proteinaceous foodstuff after steps a) and b) are enhanced compared to the untreated proteinaceous food product.

42-49. (canceled)

50. The foodstuff prepared by the method of claim 40.

51. The proteinaceous foodstuff according to claim 50,
wherein the concentration of free thiols present in said food product is no greater than 15 μM, or no greater than 10 μM, or no greater than 5 μM or
in which the gel strength has been enhanced by at least 2 g or
in which the water holding capacity has been enhanced by at least 5%, such as at least 10% or
in which at least 10% of the total myosin is cross linked or
in which the concentration of free thiols has been reduced by at least 2 μM or
in which the proteinaceous foodstuff is derived from animal meat, such as mammalian, bird, or fish or
in which the food product is free from artificial additives, particularly additives such as phosphates.

52-57. (canceled)

58. A processed food product comprising the proteinaceous foodstuff according to claim 50.

59. The processed food product according to claim 58, wherein the food product is selected from the group consisting of cooked meat, dry & semi-dry cured meat products, fermented products, emulsified products, fish & seafood products, fresh meat muscle, ground/restructered fresh meat, reformed meat, poultry products, retorted products, autoclaved meat products, vegetarian & meat analogue products.

60-62. (canceled)
